(19) United States
(12) Patent Application Publication
(10) Pub. No.: US 2022/0096517 A1
(43) Pub. Date: Mar. 31, 2022

(54) MicroRNA Compounds and Methods for Modulating MIR-10B Activity

(71) Applicant: Regulus Therapeutics Inc., San Diego, CA (US)

(72) Inventor: Charles R. Allerson, San Diego, CA (US)

(73) Assignee: Regulus Therapeutics Inc., San Diego, CA (US)

(21) Appl. No.: 17/290,298

(22) PCT Filed: Nov. 12, 2019
(86) PCT No.: PCT/US2019/060841

§ 371 Date: Apr. 30, 2021

Related U.S. Application Data
(60) Provisional application No. 62/760,546, filed on Nov. 13, 2018.

(51) Int. Cl. A61K 31/712; A61K 31/4188; A61K 31/7125; A61P 35/00; C12N 15/113; A61K 45/06

(52) U.S. Cl. CPC A61K 31/712; A61K 31/4188; A61K 31/7125; A61K 2300/00; C12N 15/113; A61K 45/06; A61P 35/00

(57) ABSTRACT

Described herein are compositions and methods for the inhibition of miR-10b activity. The compositions may be administered to subjects with cancer, such as glioma.

### CROSS-REFERENCE TO RELATED APPLICATIONS

This application is a national stage application of International Application No. PCT/US2019/060841, filed Nov. 12, 2019, which claims the benefit of priority of U.S. Provisional Application No. 62/760,546, filed Nov. 13, 2018, each of which is incorporated by reference herein in its entirety for any purpose.

### SEQUENCE LISTING

This application includes a Sequence Listing in .txt format. The .txt file contains a replacement Sequence Listing entitled “2021-10-28_01138-0040-00US_Seq_List_ST25” created on Oct. 28, 2021, and is 5,585 bytes in size. The Sequence Listing contained in this .txt file is part of the specification and is hereby incorporated by reference herein in its entirety.

### FIELD OF INVENTION

Provided herein are methods and compositions for the modulation of miR-10b activity.

### DESCRIPTION OF RELATED ART

MicroRNAs (microRNAs), also known as “mature microRNA” are small (approximately 18-24 nucleotides in length), non-coding RNA molecules encoded in the genomes of plants and animals. In certain instances, highly conserved, endogenously expressed microRNAs regulate the expression of genes by binding to the 3′-untranslated regions (3′-UTR) of specific mRNAs. More than 1000 different microRNAs have been identified in plants and animals. Certain mature microRNAs appear to originate from long endogenous primary microRNA transcripts (also known as pri-microRNAs, pri-mirs, pri-miRs or pri-pre-microRNAs) that are often hundreds of nucleotides in length (Lee, et al., EMBO J., 2002, 21(17), 4663-4670).

Functional analyses of microRNAs have revealed that these small non-coding RNAs contribute to different physiological processes in animals, including developmental timing, organogenesis, differentiation, patterning, embryogenesis, growth control and programmed cell death. Examples of particular processes in which microRNAs participate include stem cell differentiation, neurogenesis, angiogenesis, hematopoiesis, and exocytosis (reviewed by Alvarez-Garcia and Miska, Development, 2005, 132, 4653-4662).

MicroRNAs have also been associated with carcinogenesis by targeting tumor suppressors (see Gabriely et al., Cancer Res. 2011, 71(10): 3563-3572). For example, miR-10b is a powerful oncogenic microRNA associated with poor prognosis in a variety of cancers (see Teplyuk N et al., EMBO Molecular Medicine, 2016, 8(3), 268-287). Based on the cancer type and genetic context, miR-10b can promote proliferation, survival, and migration of tumor cells by directly targeting a variety of genes. In particular, miR-10b has been reported to regulate invasion and metastasis of breast cancer and squamous cell carcinoma cells.

A unique property of miR-10b is that it is highly expressed in gliomas (i.e., primary brain cancer that grows from glial cells), but is absent in normal neuroglial cells. In cultured glioma cells, miR-10b regulates cell cycle and alternative splicing in target genes (see Teplyuk 2016).

Glioblastoma, which may also be referred to as grade IV astrocytoma, is the highest grade malignant glioma and the most common malignant primary brain tumor in adults. Glioblastoma patients have a median survival of approximately 14 months due to a lack of effective treatments. Approximately 90% of glioblastomas cases exhibit high expression of miR-10b, supporting its potential role in tumor development. The profile of high expression of miR-10b in gliomas and its absence in normal neuroglial cells suggest that therapies targeting miR-10b could be effective treatments for gliomas.

### SUMMARY OF INVENTION

Embodiment 1. A compound comprising a modified oligonucleotide, wherein modified oligonucleotide consists of 21 linked nucleosides and the structure of the modified oligonucleotide is:

(SEQ ID NO: 2)
5′-C_{K}A_{K}A_{K}AU_{K}U_{K}C_{K}GG_{K}U_{E}U_{E}C_{E}U_{E}A_{E}C_{E}A_{E}G_{E}G_{E}G_{E}U_{E}A_{E}-3′

wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein each U is independently selected from a non-methylated uracil and a 5-methyluracil; wherein each C is independently selected from a non-methylated cytosine and a 5-methylcytosine; and wherein each linkage is a phosphorothioate linkage; or a pharmaceutically acceptable salt thereof.

Embodiment 2. The compound of embodiment 1, wherein the modified oligonucleotide consists of 21 linked nucleosides and the structure of the modified oligonucleotide is:

(SEQ ID NO: 2)
5′-C_{K}A_{K}A_{K}AU_{K}U_{K}C_{K}GG_{K}^{m}U_{E}^{m}U_{E}^{m}C_{E}^{m}U_{E}A_{E}^{m}C_{E}A_{E}G_{E}G_{E}G_{E}^{m}U_{E}A_{E}-3′

wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein a “^{m}U” is a 5-methyluracil and “U” is a non-methylated uracil; wherein a “^{m}C” is a 5-methylcytosine and “C” is a non-methylated cytosine; and wherein each linkage is a phosphorothioate linkage; or a pharmaceutically acceptable salt thereof.

Embodiment 3. A compound comprising a modified oligonucleotide, wherein modified oligonucleotide consists of 21 linked nucleosides and the structure of the modified oligonucleotide is:

(SEQ ID NO: 2)
5′-C_{K}A_{K}A_{E}A_{E}U_{K}U_{E}C_{E}G_{K}G_{E}U_{E}U_{K}C_{E}U_{E}A_{K}C_{E}A_{E}G_{E}G_{E}G_{E}U_{E}A_{E}-3′

wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein each U is independently selected from a non-methylated uracil and a 5-methyluracil; wherein each C is independently selected from a non-methylated cytosine and a 5-methylcytosine; and wherein each linkage is a phosphorothioate linkage; or a pharmaceutically acceptable salt thereof.

Embodiment 4. The compound of embodiment 3, wherein modified oligonucleotide consists of 21 linked nucleosides and the structure of the modified oligonucleotide is:

(SEQ ID NO: 2)
5′-C_{K}A_{K}A_{E}A_{E}U_{K}^{m}U_{E}^{m}C_{E}G_{K}G_{E}^{m}U_{E}U_{K}^{m}C_{E}^{m}U_{E}A_{K}^{m}C_{E}A_{E}G_{E}G_{E}G_{E}^{m}U_{E}A_{E}-3′

wherein nucleosides followed by subscript “E” are 2′-O-methoxyethyl nucleosides, and nucleosides followed by subscript “K” are S-cEt nucleosides; wherein a “^{m}U” is a 5-methyluracil and “U” is a non-methylated uracil; wherein a “^{m}C” is a 5-methylcytosine; and wherein each internucleoside linkage is a phosphorothioate linkage; or a pharmaceutically acceptable salt thereof.

Embodiment 5. A compound comprising a modified oligonucleotide consisting of 9 linked nucleosides, wherein the modified oligonucleotide comprises the structure:

5′-U_{K}A_{K}C_{M}A_{F}G_{F}G_{F}G_{M}U_{K}A_{K}-3′

wherein nucleosides followed by subscript “K” are S-cEt nucleosides, nucleosides followed by subscript “M” are 2′-O-methyl nucleosides, and nucleosides followed by subscript “F” are 2′-fluoro nucleosides; wherein each U is independently selected from a non-methylated uracil and a 5-methyluracil; wherein each C is independently selected from a non-methylated cytosine and a 5-methylcytosine; and wherein each internucleoside linkage is a phosphorothioate linkage; or a pharmaceutically acceptable salt thereof.

Embodiment 6. The compound of embodiment 7, wherein the modified oligonucleotide consists of 9 linked nucleosides and the structure of the modified oligonucleotide is:

5′-U_{K}A_{K}C_{M}A_{F}G_{F}G_{F}G_{M}U_{K}A_{K}-3′

wherein nucleosides followed by subscript “K” are S-cEt nucleosides nucleosides followed by subscript “M” are 2′-O-methyl nucleosides, and nucleosides followed by subscript “F” are 2′-fluoro nucleosides; wherein a “U” is a non-methylated uracil; wherein a “C” is a non-methylated cytosine; wherein a superscript “0” indicates a phosphodiester linkage and each other internucleoside linkage is a phosphorothioate linkage; or a pharmaceutically acceptable salt thereof.

Embodiment 7. The compound of any one of embodiments 1 to Error! Reference source not found., wherein the compound consists of the modified oligonucleotide, or a pharmaceutically acceptable salt thereof.

Embodiment 8. The compound of any one of embodiments 1 to Error! Reference source not found., wherein the pharmaceutically acceptable salt is a sodium salt.

Embodiment 9. A pharmaceutical composition comprising a compound of any one of embodiments to 1 to Error! Reference source not found., and a pharmaceutically acceptable diluent.

Embodiment 10. The pharmaceutical composition of embodiment Error! Reference source not found., wherein the pharmaceutically acceptable diluent is an aqueous solution.

Embodiment 11. The pharmaceutical composition of embodiment Error! Reference source not found., wherein the aqueous solution is a saline solution.

Embodiment 12. A pharmaceutical composition comprising a compound of any one of embodiments 1 to Error! Reference source not found., which is a lyophilized composition.

Embodiment 13. A pharmaceutical composition consisting essentially of a compound of any one of embodiments 1 to Error! Reference source not found. in a saline solution.

Embodiment 14. A method of treating glioma, comprising administering to a subject having glioma a compound of any one of embodiments 1 to 6, or a pharmaceutical composition of any one of embodiments Error! Reference source not found. to Error! Reference source not found. or Error! Reference source not found.

Embodiment 15. The method of embodiment Error! Reference source not found., wherein the glioma is diffuse astrocytoma, anaplastic astrocytoma, oligodendroglioma, anaplastic oligodendroglioma, diffuse midline glioma, or glioblastoma.

Embodiment 16. The method of embodiment 14 or 15, wherein the compound or pharmaceutical composition is administered intratumorally.

Embodiment 17. The method of embodiment Error! Reference source not found., wherein the diffuse astrocytoma comprises an isocitrate dehydrogenase (IDH) gene mutation.

Embodiment 18. The method of embodiment Error! Reference source not found., wherein the anaplastic astrocytoma comprises an isocitrate dehydrogenase (IDH) gene mutation.

Embodiment 19. The method of embodiment Error! Reference source not found., wherein the oligodendroglioma comprises an isocitrate dehydrogenase (IDH) gene mutation and a deletion of chromosomal arms 1p and 19q.

Embodiment 20. The method of embodiment Error! Reference source not found., wherein the anaplastic oligodendroglioma comprises an isocitrate dehydrogenase (IDH) gene mutation and a deletion of chromosomal arms 1p and 19q.

Embodiment 21. The method of embodiment Error! Reference source not found., wherein the diffuse midline glioma comprises a comprises a histone H3 (H3) K27M mutation.

Embodiment 22. The method of embodiment Error! Reference source not found., wherein the glioblastoma does not comprise an isocitrate dehydrogenase (IDH) gene mutation.

Embodiment 23. The method of embodiment Error! Reference source not found., wherein the glioblastoma comprises an isocitrate dehydrogenase (IDH) gene mutation.

Embodiment 24. The method of any one of embodiment Error! Reference source not found. to Error! Reference source not found., wherein the glioma is a recurrent glioma.

Embodiment 25. The method of any one of embodiments 17, Error! Reference source not found., Error! Reference source not found., Error! Reference source not found., 22 or Error!Reference source not found., wherein the isocitrate dehydrogenase (IDH) gene mutation is an IDH1 or IDH2 gene mutation.

Embodiment 26. The method of any one of embodiments Error! Reference source not found. to 25, wherein following administration of the compound or pharmaceutical composition, tumor size is reduced and/or tumor number is reduced.

Embodiment 27. The method of any one of embodiments Error! Reference source not found. to Error! Reference source not found., wherein the administering of the compound or pharmaceutical composition increases progression-free survival of the subject.

Embodiment 28. The method of any one of embodiments Error! Reference source not found. to Error! Reference source not found., wherein the administering of the compound or pharmaceutical composition increases overall survival time of the subject.

Embodiment 29. The method of any one of embodiments Error! Reference source not found. to Error! Reference source not found., wherein the administering of the compound improves the subject's quality of life.

Embodiment 30. The method of any one of embodiments Error! Reference source not found. to Error! Reference source not found., comprising administering at least one additional anti-cancer therapy.

Embodiment 31. The method of embodiment Error! Reference source not found., wherein the at least one additional therapy is selected from surgical resection, radiotherapy, tumor treating fields, and one or more chemotherapeutic agents.

Embodiment 32. The method of embodiment Error! Reference source not found., wherein the chemotherapeutic agent is selected from carmustine, temozolomide, and bevacizumab.

Embodiment 33. The method of embodiment Error! Reference source not found., wherein the chemotherapeutic agent is temozolomide.

Embodiment 34. The method of embodiment Error! Reference source not found., wherein the at least one additional anti-cancer therapy comprises surgical resection, radiotherapy, and temozolomide.

### BRIEF DESCRIPTION OF THE DRAWING

FIG. 1 shows percent survival of glioblastoma multiforme (GBM) model mice administered RG5579 alone, temozolomide (TMZ) alone, or the combination of RG5579 and TMZ.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sanghvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Glioma” means a primary brain cancer that grows from glial cells. In certain embodiments, glioma includes, but is not limited to, cancer arising from astrocytes, such as, for example, astrocytomas; cancer arising from oligodendrocytes, such as, for example, oligodendroglioma; and cancers of mixed origin, such as oligoastrocytomas. The term “glioma” also includes glioblastoma (or glioblastoma multiforme (GBM)), which is a malignant glioma.

“Metastasis” means the process by which cancer spreads from the place at which it first arose as a primary tumor to other locations in the body. The metastatic progression of a primary tumor reflects multiple stages, including dissociation from neighboring primary tumor cells, survival in the circulation, and growth in a secondary location.

“Overall survival time” means the period for which a subject survives after diagnosis of or treatment for a disease. In certain embodiments, the disease is cancer. In some embodiments, overall survival time is survival after diagnosis. In some embodiments, overall survival time is survival after the start of treatment.

“Progression-free survival” means the period for which a subject having a disease survives, without the disease getting worse. In certain embodiments, progression-free survival is assessed by staging or scoring the disease. In certain embodiments, progression-free survival of a subject having liver cancer is assessed by evaluating tumor size, tumor number, and/or metastasis.

“Halts further progression” means to stop movement of a medical condition to an advanced state.

“Slows further progression” means to reduce the rate at which a medical condition moves towards an advanced state.

“Improves life expectancy” means to lengthen the life of a subject by treating one or more symptoms of a disease in the subject.

“Quality of life” means the extent to which a subject's physical, psychological, and social functioning are impaired by a disease and/or treatment of a disease.

“Anti-miR” means an oligonucleotide having a nucleobase sequence complementary to a microRNA. In certain embodiments, an anti-miR is a modified oligonucleotide.

“Anti-miR-Ob” means a modified oligonucleotide having a nucleobase sequence complementary to miR-10b. In certain embodiments, an anti-miR-10b is fully complementary to miR-10b (i.e., 100% complementary). In certain embodiments, an anti-miR-10b is at least 80%, at least 85%, at least 90%, or at least 95% complementary to miR-10b.

“miR-10b” means the mature miRNA having the nucleobase sequence

(SEQ ID NO: 1)
UACCCUGUAGAACCGAAUUUGUG.

“miR-10b seed sequence” means the nucleobase sequence 5′-ACCCUG-3′, which is present in miR-10b.

“Subject in need thereof” means a subject that is identified as in need of a therapy or treatment.

“Subject suspected of having” means a subject exhibiting one or more clinical indicators of a disease.

“Disease associated with miR-10b” means a disease or condition that is modulated by the activity of miR-10b.

“Administering” means providing a pharmaceutical agent or composition to a subject, and includes, but is not limited to, administering by a medical professional and self-administering.

“Parenteral administration” means administration through injection or infusion.

Parenteral administration includes, but is not limited to, subcutaneous administration, intravenous administration, and intramuscular administration.

“Subcutaneous administration” means administration just below the skin.

“Intravenous administration” means administration into a vein.

“Administered concomitantly” refers to the co-administration of two or more agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period and need not be coextensive.

“Duration” means the period during which an activity or event continues. In certain embodiments, the duration of treatment is the period during which doses of a pharmaceutical 1

“Therapy” means a disease treatment method. In certain embodiments, therapy includes, but is not limited to, chemotherapy, radiation therapy, or administration of a pharmaceutical agent.

“Treatment” or “treat” means the application of one or more specific procedures used for the cure or amelioration of a disease. In certain embodiments, the specific procedure is the administration of one or more pharmaceutical agents.

“Ameliorate” means to lessen the severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“At risk for developing” means the state in which a subject is predisposed to developing a condition or disease. In certain embodiments, a subject at risk for developing a condition or disease exhibits one or more symptoms of the condition or disease, but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

“Prevent the onset of” means to prevent the development of a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to an animal.

“Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Pharmaceutically acceptable salt” means a physiologically and pharmaceutically acceptable salt of a compound provided herein, i.e., a salt that retains the desired biological activity of the compound and does not have undesired toxicological effects when administered to a subject. Nonlimiting exemplary pharmaceutically acceptable salts of compounds provided herein include sodium and potassium salt forms. The terms “compound,” “oligonucleotide,” and “modified oligonucleotide” as used herein include pharmaceutically acceptable salts thereof unless specifically indicated otherwise.

“Saline solution” means a solution of sodium chloride in water.

“Improved organ function” means a change in organ function toward normal limits. In certain embodiments, organ function is assessed by measuring molecules found in a subject's blood or urine. For example, in certain embodiments, improved liver function is measured by a reduction in blood liver transaminase levels. In certain embodiments, improved kidney function is measured by a reduction in blood urea nitrogen, a reduction in proteinuria, a reduction in albuminuria, etc.

“Acceptable safety profile” means a pattern of side effects that is within clinically acceptable limits.

“Side effect” means a physiological response attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

The term “blood” as used herein, encompasses whole blood and blood fractions, such as serum and plasma.

“Target nucleic acid” means a nucleic acid to which an oligomeric compound is designed to hybridize.

“Targeting” means the process of design and selection of nucleobase sequence that will hybridize to a target nucleic acid.

“Targeted to” means having a nucleobase sequence that will allow hybridization to a target nucleic acid.

“Target engagement” means the interaction of an oligonucleotide with the microRNA to which it is complementary, in a manner that changes the activity, expression or level of the microRNA. In certain embodiments, target engagement means an anti-miR interacting with the microRNA to which it is complementary, such that the activity of the microRNA is inhibited.

“Modulation” means a perturbation of function, amount, or activity. In certain embodiments, modulation means an increase in function, amount, or activity. In certain embodiments, modulation means a decrease in function, amount, or activity.

“Expression” means any functions and steps by which a gene's coded information is converted into structures present and operating in a cell.

“Nucleobase sequence” means the order of contiguous nucleobases in an oligomeric compound or nucleic acid, typically listed in a 5′ to 3′ orientation, and independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that one nucleic acid is capable of hybridizing to another nucleic acid or oligonucleotide. In certain embodiments, complementary refers to an oligonucleotide capable of hybridizing to a target nucleic acid.

“Fully complementary” means each nucleobase of an oligonucleotide is capable of pairing with a nucleobase at each corresponding position in a target nucleic acid. In certain embodiments, an oligonucleotide is fully complementary (also referred to as 100% complementary) to a microRNA, i.e. each nucleobase of the oligonucleotide is complementary to a nucleobase at a corresponding position in the microRNA. A modified oligonucleotide may be fully complementary to a microRNA, and have a number of linked nucleosides that is less than the length of the microRNA. For example, an oligonucleotide with 16 linked nucleosides, where each nucleobase of the oligonucleotide is complementary to a nucleobase at a corresponding position in a microRNA, is fully complementary to the microRNA.

“Percent complementarity” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Percent identity” means the number of nucleobases in a first nucleic acid that are identical to nucleobases at corresponding positions in a second nucleic acid, divided by the total number of nucleobases in the first nucleic acid. In certain embodiments, the first nucleic acid is a microRNA and the second nucleic acid is a microRNA. In certain embodiments, the first nucleic acid is an oligonucleotide and the second nucleic acid is an oligonucleotide.

“Hybridize” means the annealing of complementary nucleic acids that occurs through nucleobase complementarity.

“Mismatch” means a nucleobase of a first nucleic acid that is not capable of Watson-Crick pairing with a nucleobase at a corresponding position of a second nucleic acid.

“Identical” in the context of nucleobase sequences, means having the same nucleobase sequence, independent of sugar, linkage, and/or nucleobase modifications and independent of the methylation state of any pyrimidines present.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (microrna.sanger.ac.uk/). In certain embodiments, microRNA is abbreviated as “miR.”

“microRNA-regulated transcript” means a transcript that is regulated by a microRNA.

“Seed match sequence” means a nucleobase sequence that is complementary to a seed sequence, and is the same length as the seed sequence.

“Oligomeric compound” means a compound that comprises a plurality of linked monomeric subunits. Oligomeric compounds include oligonucleotides.

“Oligonucleotide” means a compound comprising a plurality of linked nucleosides, each of which can be modified or unmodified, independent from one another.

“Naturally occurring internucleoside linkage” means a 3′ to 5′ phosphodiester linkage between nucleosides.

“Natural sugar” means a sugar found in DNA (2′-H) or RNA (2′-OH).

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

“Compound comprising a modified oligonucleotide consisting of” a number of linked nucleosides means a compound that includes a modified oligonucleotide having the specified number of linked nucleosides. Thus, the compound may include additional substituents or conjugates. Unless otherwise indicated, the modified oligonucleotide is not hybridized to a complementary strand and the compound does not include any additional nucleosides beyond those of the modified oligonucleotide.

“Modified oligonucleotide” means a single-stranded oligonucleotide having one or more modifications relative to a naturally occurring terminus, sugar, nucleobase, and/or internucleoside linkage. A modified oligonucleotide may comprise unmodified nucleosides.

“Modified nucleoside” means a nucleoside having any change from a naturally occurring nucleoside. A modified nucleoside may have a modified sugar, and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

“Modified internucleoside linkage” means any change from a naturally occurring internucleoside linkage.

“Phosphorothioate internucleoside linkage” means a linkage between nucleosides where one of the non-bridging atoms is a sulfur atom.

“Modified sugar moiety” means substitution and/or any change from a natural sugar.

“Unmodified nucleobase” means the naturally occurring heterocyclic bases of RNA or DNA: the purine bases adenine (A) and guanine (G), and the pyrimidine bases thymine (T), cytosine (C) (including 5-methylcytosine), and uracil (U).

“5-methylcytosine” means a cytosine comprising a methyl group attached to the 5 position.

“Non-methylated cytosine” means a cytosine that does not have a methyl group attached to the 5 position.

“5-methyluracil” means a uracil comprising a methyl group attached to the 5 position. A 5-methyluracil may also be referred to a thymine.

“Non-methylated uracil” means a uracil that does not have a methyl group attached to the 5 position.

“Modified nucleobase” means any nucleobase that is not an unmodified nucleobase.

“Sugar moiety” means a naturally occurring furanosyl or a modified sugar moiety.

“Modified sugar moiety” means a substituted sugar moiety or a sugar surrogate.

“2′-O-methyl sugar” or “2′-OMe sugar” means a sugar having an O-methyl modification at the 2′ position.

“2′-O-methoxyethyl sugar” or “2′-MOE sugar” means a sugar having an O-methoxyethyl modification at the 2′ position.

“2′-fluoro” or “2′-F” means a sugar having a fluoro modification of the 2′ position.

“Bicyclic sugar moiety” means a modified sugar moiety comprising a 4 to 7 membered ring (including by not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl. Nonlimiting exemplary bicyclic sugar moieties include LNA, ENA, cEt, S-cEt, and R-cEt.

“Locked nucleic acid (LNA) sugar moiety” means a substituted sugar moiety comprising a (CH₂)—O bridge between the 4′ and 2′ furanose ring atoms.

“ENA sugar moiety” means a substituted sugar moiety comprising a (CH₂)₂—O bridge between the 4′ and 2′ furanose ring atoms.

“Constrained ethyl (cEt) sugar moiety” means a substituted sugar moiety comprising a CH(CH₃)—O bridge between the 4′ and the 2′ furanose ring atoms. In certain embodiments, the CH(CH₃)—O bridge is constrained in the S orientation. In certain embodiments, the CH(CH₃)—O is constrained in the R orientation.

“S-cEt sugar moiety” means a substituted sugar moiety comprising an S-constrained CH(CH₃)—O bridge between the 4′ and the 2′ furanose ring atoms.

“R-cEt sugar moiety” means a substituted sugar moiety comprising an R-constrained CH(CH₃)—O bridge between the 4′ and the 2′ furanose ring atoms.

“2′-O-methyl nucleoside” means a 2′-modified nucleoside having a 2′-O-methyl sugar modification.

“2′-O-methoxyethyl nucleoside” means a 2′-modified nucleoside having a 2′-O-methoxyethyl sugar modification. A 2′-O-methoxyethyl nucleoside may comprise a modified or unmodified nucleobase.

“2′-fluoro nucleoside” means a 2′-modified nucleoside having a 2′-fluoro sugar modification. A 2′-fluoro nucleoside may comprise a modified or unmodified nucleobase.

“Bicyclic nucleoside” means a 2′-modified nucleoside having a bicyclic sugar moiety. A bicyclic nucleoside may have a modified or unmodified nucleobase.

“cEt nucleoside” means a nucleoside comprising a cEt sugar moiety. A cEt nucleoside may comprise a modified or unmodified nucleobase.

“S-cEt nucleoside” means a nucleoside comprising an S-cEt sugar moiety.

“R-cEt nucleoside” means a nucleoside comprising an R-cEt sugar moiety.

“β-D-deoxyribonucleoside” means a naturally occurring DNA nucleoside.

“β-D-ribonucleoside” means a naturally occurring RNA nucleoside. “LNA nucleoside” means a nucleoside comprising a LNA sugar moiety.

“ENA nucleoside” means a nucleoside comprising an ENA sugar moiety.

“Subject” means a human or non-human animal selected for treatment or therapy.

### Overview

It is estimated that more than 1.6 million Americans are diagnosed with cancer each year. Even with improvements in screening and treatment, cancer remains the second leading cause of death in the United States after heart disease.

MicroRNAs can promote carcinogenesis by targeting tumor suppressors that regulate cell cycle and apoptosis. For example, miR-10b is an oncogenic microRNA that can regulate invasion, migration, and metastasis of cells from a variety of different cancers. In particular, miR-10b is highly expressed in all subtypes of glioblastoma, but is absent from normal neuroglial cells. miR-10b regulates cell cycle and alternative splicing in glioma cells, and inhibition of miR-10b is associated with impaired proliferation and survival of these cells.

Gliomas, and particularly glioblastoma, continue to have significant unmet medical need. Current treatments for glioblastoma are associated with significant toxicity and very high rates of recurrence. Even with intensive treatment, the median survival of glioblastoma patients is approximately 14 months. Thus, while an unmet need is present for all cancers, gliomas in particular are a cancer with significant burden and need for improved treatments. Treatments aimed at inhibiting miR-10b are therefore of high interest for treatment of gliomas.

As such, these compounds are useful for the modulation of cellular processes that are promoted by the activity of miR-10b. Further, such compounds are useful for treating, preventing, and/or delaying the onset of diseases associated with miR-10b. Such diseases may be characterized by abnormally high expression of miR-10b, relative to non-disease samples. Such diseases include, but are not limited to, cancer, including gliomas.

To identify anti-miR-10b compounds that are sufficiently efficacious, convenient and safe to administer to subjects with cancer, such as glioma, approximately 215 modified oligonucleotides targeted to miR-10b were designed, having varying lengths and chemical composition. The length of the compounds ranged from 9 to 23 linked nucleosides, and the compounds varied in the number, type, and placement of chemical modifications. As pharmacology, pharmacokinetic behavior and safety cannot be predicted simply based on a compound's chemical structure, compounds were evaluated both in vitro and in vivo for characteristics including potency, efficacy, pharmacokinetic behavior, safety, and metabolic stability, in a series of assays designed to eliminate compounds with unfavorable properties. As described herein, the approximately 215 compounds were first tested in several in vitro assays (e.g. potency, toxicology, metabolic stability), to identify a smaller set of compounds suitable for further testing in more complex in vivo assays (e.g. pharmacokinetic profile, efficacy, toxicology). This screening process identified candidate pharmaceutical agents for the treatment of cancer, including glioma.

Certain Modified Oligonucleotides Targeted to miR-10b

Provided herein are compounds comprising modified oligonucleotides targeted to miR-10b.

In certain embodiments, the modified oligonucleotide consists of 21 linked nucleosides and the structure of the modified oligonucleotide is:

(SEQ ID NO: 2)
5′- C_{K}A_{K}A_{K}AU_{K}U_{K}C_{K}GG_{K}U_{E}U_{E}C_{E}U_{E}A_{E}C_{E}A_{E}G_{E}G_{E}G_{E}U_{E}A_{E} -3′

wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein each U is independently selected from a non-methylated uracil and a 5-methyluracil; wherein each C is independently selected from a non-methylated cytosine and a 5-methylcytosine; and wherein each linkage is a phosphorothioate linkage; or a pharmaceutically acceptable salt thereof.

In certain embodiments, the modified oligonucleotide consists of 21 linked nucleosides and the structure of the modified oligonucleotide is:

(SEQ ID NO: 2)
5′- C_{K}A_{K}A_{K}AU_{K}U_{K}C_{K}GG_{K}^{m}U_{E}^{m}U_{E}^{m}C_{E}^{m}U_{E}A_{E}^{m}C_{E}A_{E}G_{E}G_{E}G_{E}^{m}U_{E}
A_{E}-3′

wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein a “^{m}U” is a 5-methyluracil and “U” is a non-methylated uracil; wherein a “^{m}C” is a 5-methylcytosine and “C” is a non-methylated cytosine; and wherein each linkage is a phosphorothioate linkage; or a pharmaceutically acceptable salt thereof.

In certain embodiments, the modified oligonucleotide consists of 21 linked nucleosides, wherein the modified oligonucleotide comprises the structure:

(SEQ ID NO: 2)
5′-C_{K}A_{K}A_{E}A_{E}U_{K}U_{E}^{m}C_{E}G_{K}G_{E}U_{E}U_{K}^{m}C_{E}U_{E}A_{K}^{m}C_{E}A_{E}G_{E}G_{E}G_{E}U_{E}A_{E}-
3′

wherein nucleosides followed by subscript “E” are 2′-O-methoxyethyl nucleosides, and nucleosides followed by subscript “K” are S-cEt nucleosides; wherein each U is independently selected from a non-methylated uracil and a 5-methyluracil; wherein each C is independently selected from a non-methylated cytosine and a 5-methylcytosine; and wherein each internucleoside linkage is a phosphorothioate linkage; or a pharmaceutically acceptable salt thereof.

In certain embodiments, the modified oligonucleotide consists of 21 linked nucleosides and the structure of the modified oligonucleotide is:

(SEQ ID NO: 2)
5′-C_{K}A_{K}A_{E}A_{E}U_{K}^{m}U_{E}mC_{E}G_{K}G_{E}^{m}U_{E}U_{K}^{m}C_{E}^{m}U_{E}A_{K}^{m}C_{E}A_{E}G_{E}G_{E}G_{E}^{m}U_{E}
A_{E}-3′

wherein nucleosides followed by subscript “E” are 2′-O-methoxyethyl nucleosides, and nucleosides followed by subscript “K” are S-cEt nucleosides; wherein a “^{m}U” is a 5-methyluracil and “U” is a non-methylated uracil; wherein a “^{m}C” is a 5-methylcytosine; and wherein each internucleoside linkage is a phosphorothioate linkage; or a pharmaceutically acceptable salt thereof.

In certain embodiments, modified oligonucleotide consisting of 9 linked nucleosides, wherein the modified oligonucleotide comprises the structure:

5′-U_{K}A_{K}C_{M}A_{F}G_{F}G_{F}G_{M}U_{K}A_{K}-3′

wherein nucleosides followed by subscript “M” are 2′-O-methyl nucleosides, nucleosides followed by subscript “F” are 2′-fluoro nucleosides, and nucleosides followed by subscript “K” are S-cEt nucleosides; wherein each U is independently selected from a non-methylated uracil and a 5-methyluracil; wherein each C is independently selected from a non-methylated cytosine and a 5-methylcytosine; and each internucleoside linkage is a phosphorothioate linkage; or a pharmaceutically acceptable salt thereof.

In certain embodiments, the modified oligonucleotide consists of 9 linked nucleosides and the structure of the modified oligonucleotide is:

5′-U_{K}A_{K}C_{M}A_{F}G_{F}G_{F}G_{M}U_{K}A_{K}-3′

wherein nucleosides followed by subscript “M” are 2′-O-methyl nucleosides, nucleosides followed by subscript “F” are 2′-fluoro nucleosides, and nucleosides followed by subscript “K” are S-cEt nucleosides; wherein each U is a non-methylated uracil; wherein each C is a non-methylated cytosine; and each internucleoside linkage is a phosphorothioate linkage; or a pharmaceutically acceptable salt thereof.

Provided herein are pharmaceutically acceptable salts of modified oligonucleotides. In certain embodiments, the pharmaceutically acceptable salt is a sodium salt.

In some embodiments, a pharmaceutically acceptable salt of a modified oligonucleotide comprises fewer cationic counterions (such as Na⁺) than there are phosphorothioate and/or phosphodiester linkages per molecule (i.e., some phosphorothioate and/or phosphodiester linkages are protonated). In some embodiments, a pharmaceutically acceptable salt of a modified oligonucleotide comprises fewer than 17 cationic counterions (such as Na⁺) per molecule of modified oligonucleotide. That is, in some embodiments, a pharmaceutically acceptable salt of a modified oligonucleotide may comprise, on average, 1, 2, 3, 4, 5, 6, 7, 8, 9, 10, 11, 12, 13, 14, 15, 16, 17, 18, 19, or 20 cationic counterions per molecule of modified oligonucleotide, with the remaining phosphorothioate and/or phosphodiester groups being protonated.

Further provided are compounds comprising any of the modified oligonucleotides described herein.

### Certain Uses of the Invention

Provided herein are methods for inhibiting the activity of miR-10b in a cell, comprising contacting a cell with a compound provided herein, which comprises a nucleobase sequence complementary to the miR-10b. In certain embodiments, the cell is a cancer cell. In certain embodiments, the cell is a glioma cell.

In certain embodiments, contacting a cancer cell with a compound provided herein induces apoptosis in the cancer cell. In certain embodiments, contacting a cancer cell with a compound provided herein decreases cell proliferation.

Provided herein are methods for inhibiting the activity of miR-10b, comprising administering to the subject a pharmaceutical composition provided herein. In certain embodiments, the subject has a disease associated with miR-10b. In certain embodiments, the disease associated with miR-10b is glioma.

Provided herein are methods for the treatment of glioma in a subject, comprising administering to a subject having glioma a compound provided herein, which comprises a nucleobase sequence complementary to miR-10b.

A glioma is a cancer of the brain that arises from glial cells. Glial cells include astrocytes, oligodendrocytes, microglia and ependymal cells, which together function to provide energy and nutrients to nerve cells, in addition to maintaining the blood-brain barrier. Gliomas are classified based on genetic and/or histological features. Genetic features include, but are not limited to chromosomal loss, chromosomal translocation, chromosomal amplification, and gene mutation. For example, a glioma may be characterized by deletion of chromosomal arms 1p and 19q, and/or by a mutation in the isocitrate dehydrogenase (IDH) gene. Histopathological features include cell type of origin, expression of lineage-associated proteins, ultrastructural characterization, and level of differentiation. For example, a glioma may be identified as diffuse (widely spread) or as anaplastic (poorly differentiated). In certain cases, a glioma may not be classified into a specifically defined genetic group, for example, if insufficient genetic information is avaibable. In such cases, the glioma is given a designation of “not otherwise specified (NOS).”

Gliomas may be further graded based on how rapidly the glial tumor cells are dividing, and on the likelihood that the cells will infiltrate nearby tissues. Gliomas are assigned a grade of I, II, III, or IV, ranging from least to most aggressive.

In certain embodiments, gliomas are classified based on the World Health Organization (WHO) classification system and grading system for tumors of the central nervous system (Louis et al., Acta Neuropath, 2016, 131:803-820).

In certain embodiments, a glioma arises from astrocytes, and is classified as an astrocytoma. In certain embodiments, a glioma arises from oligodendrocytes, and is classified as an oligodendroglioma. In certain embodiments, a glioma is of mixed origin, arising from astrocytes and oligodendrocytes, and is classified as an oligoastrocytoma. In certain embodiments, a glioma arises from ependymal cells, and is classified as an ependymoma.

In certain embodiments, a glioma is a diffuse astrocytoma. In certain embodiments, a diffuse astrocytoma comprises an IDH gene mutation. In certain embodiments, a diffuse astrocytoma is a gemistocytic astrocytoma comprising an IDH gene mutation. In certain embodiments, a diffuse astrocytoma is classified as not otherwise specified. Diffuse astrocytoma is generally classified as a grade II glioma.

In certain embodiments, a glioma is an anaplastic astrocytoma. In certain embodiments, an anaplastic astrocytoma comprises an IDH gene mutation. In certain embodiments, an anaplastic astrocytoma is classified as not otherwise specified. Anaplastic astrocytoma is generally classified as a grade III glioma.

In certain embodiments, a glioma is a glioblastoma. In certain embodiments, a glioblastoma does not comprise an IDH gene mutation. In certain embodiments, a glioblastoma is a giant cell glioblastoma. In certain embodiments, a glioblastoma is a gliosarcoma. In certain embodiments, a glioblastoma is an epithelioid glioblastoma. In certain embodiments, a glioblastoma is classified as not otherwise specified.

In certain embodiments, a glioblastoma comprises an IDH gene mutation. Glioblastoma is generally classified as a grade IV glioma.

In certain embodiments, a glioma is a diffuse midline glioma. In certain embodiments, a diffuse midline glioma comprises a histone H3 (H3) K27M mutation. Diffuse midline glioma is generally classified as a grade IV glioma.

In certain embodiments, a glioma is an oligodendroglioma. In certain embodiments, an oligodendroglioma comprises an IDH gene mutation and a deletion of chromosomal arm 1p and chromosomal arm 19q. In certain embodiments, an oligodendroglioma is classified as not otherwise specified. In general, oligodendroglioma is classified as a grade II glioma.

In certain embodiments, a glioma is an anaplastic oligodendroglioma. In certain embodiments, an anaplastic oligodendroglioma comprises an IDH gene mutation and a deletion of chromosomal arm 1p and chromosomal arm 19q. In certain embodiments, an anaplastic oligodendroglioma is classified as not otherwise specified. In general, anaplastic oligodendroglioma is classified as a grade III glioma.

In certain embodiments, a glioma is an oligoastrocytoma. In certain embodiments, an oligoastrocytoma is classified as not otherwise specified.

In certain embodiments, a glioma is an anaplastic oligoastrocytoma. In certain embodiments, an anaplastic oligoastrocytoma is classified as not otherwise specified.

In certain embodiments, a glioma is a pilocytic astrocytoma. In certain embodiments, a pilocytic astrocytoma is a pilomyxoid astrocytoma. In certain embodiments, a glioma is a subependymal giant cell astrocytoma. In certain embodiments, a glioma is a pleomorphic xanthoastrocytoma. In certain embodiments, a glioma is an anaplastic pleomorphic xanthoastrocytoma. Pilocytic astrocytoma is generally classified as a grade I glioma. Subependymal giant cell astrocytoma is generally classified as a grade I glioma. Anaplastic pleomorphic xanthoastrocytoma is generally classified as a grade II glioma.

In certain embodiments, a glioma is a subependymoma. A subependymoma is generally classified as a grade I glioma.

In certain embodiments, a glioma is an anaplastic ependymoma. An anaplastic ependymoma is generally classified as a grade III glioma.

In certain embodiments, a glioma is an ependymoma. In certain embodiments, a glioma is a myxopapillary ependymoma. In certain embodiments, an ependymoma is a papillary ependymoma. In certain embodiments, an ependymoma is a clear cell ependymoma. In certain embodiments, an ependymoma is a tanycytic ependymoma. In certain embodiments, an ependymoma comprises a RELA fusion (a fusion involving open reading frame C 11orf95 and the RelA gene). An ependymoma is generally classified as a grade II glioma. A myxopapillary ependymoma is generally classified as a grade I glioma. An ependymoma comprising a RELA fusion is generally classified as a grade II or grade III glioma.

In certain embodiments, a glioma is a choroid glioma of the third ventricle. In certain embodiments, a glioma is an angiocentric glioma. In certain embodiments, a glioma is an astroblastoma. An angiocentric glioma is generally classified as a grade I glioma. A choroid glioma of the third ventricle is generally classified as a grade II glioma.

In certain embodiments, an IDH gene mutation is an IDH1 gene mutation. In certain embodiments, an IDH gene mutation is an IDH2 gene mutation.

In certain embodiments, a glioma comprises a mutation in a gene selected from one or more of the TERT, CIC, FUBP1, NOTCH1, TP53, ATRX, EGFR, CDKN2A, MDM4, PTEN, and NF1 genes.

Provided herein are compositions and methods for treating, preventing, ameliorating, and/or delaying the onset of metastasis. The metastasis may result from the migration of glioma cells from the brain to any secondary location within the body. In certain embodiments, glioma metastasizes to other central nervous system tissues, for example, the spinal cord. In certain embodiments, glioma metastasizes to tissues outside the central nervous system, for example, bone, lymph node, lung, glands, and other soft tissues.

MicroRNAs bind to and repress the expression of messenger RNAs. In certain instances, inhibiting the activity of a microRNA leads to de-repression of one or more messenger RNAs, i.e. the messenger RNA expression is increased at the level of RNA and/or protein. Provided herein are methods for modulating the expression of one or more miR-10b-regulated transcripts, comprising contacting a cell with a compound of the invention, wherein the compound comprises a modified oligonucleotide having a sequence complementary to a miR-10b.

In certain embodiments, a miR-Ob-regulated transcript is Bim, TFAP2C, CDKN1A (p21), or CDKN2A (p16), and inhibition of miR-10b results in an increase in the level of Bim, TFAP2C, CDKN1A (p21), and/or CDKN2A (p16) mRNA.

miR-10b has been linked to numerous types of cancer, in addition to glioma. Accordingly, in certain embodiments, the compounds provided herein are used for treating, preventing, ameliorating, and/or delaying the onset of cancers other than glioma. In certain embodiments, the cancer is liver cancer, breast cancer, bladder cancer, prostate cancer, bone cancer, colon cancer, lung cancer, brain cancer, hematological cancer, pancreatic cancer, head and neck cancer, cancer of the tongue, stomach cancer, skin cancer, thyroid cancer, neuroblastoma, esophageal cancer, mesothelioma, neuroblastoma, kidney cancer, testicular cancer, rectal cancer, cervical cancer, or ovarian cancer. In certain embodiments, the liver cancer is hepatocellular carcinoma. In certain embodiments, the liver cancer is due to metastasis of cancer that originated in another part of the body, for example a cancer that is due to metastasis of bone cancer, colon cancer or breast cancer. In certain embodiments, the hematological cancer is acute myelogenous leukemia, acute lymphocytic leukemia, acute monocytic leukemia, multiple myeloma, chronic lymphotic leukemia, chronic myeloid leukemia, hodgkin's lymphoma, or non-hodgkin's lymphoma. In certain embodiments, the skin cancer is melanoma. In certain embodiments, the kidney cancer is renal cell carcinoma. In certain embodiments, the breast cancer is ductal cell carcinoma in situ, invasive ductal cell carcinoma, triple negative breast cancer, medullary carcinoma, tubular carcinoma, and mucinous carcinoma. In certain embodiments, the cancer is resistant to chemotherapy.

In certain embodiments, administration of the compounds or methods provided herein result in one or more clinically desirable outcomes in a subject. Such improvements may be used to determine the extent to which a subject is responding to treatment.

In certain embodiments, a clinically desirable outcome is reduction of tumor number and/or reduction of tumor size in a subject having cancer. In certain embodiments, a clinically desirable outcome is a reduction in cancer cell number in a subject having cancer. Additional clinically desirable outcomes include the extension of overall survival time of the subject, and/or extension of progression-free survival time of the subject. In certain embodiments, administration of a compound provided herein prevents an increase in tumor size and/or tumor number. In certain embodiments, administration of a compound provided herein prevents metastatic progression. In certain embodiments, administration of a compound provided herein slows or stops metastatic progression. In certain embodiments, administration of a compound provided herein prevents the recurrence of a tumor. In certain embodiments, administration of a compound provided herein delays recurrence of a tumor. In certain embodiments, administration of a compound provided herein prevents recurrence of tumor metastasis.

In any of the methods of treatment provided herein, a compound may be administered by intratumoral injection. In any of the methods of treatment provided herein, a compound may be administered by intracerebroventricular injection.

Any of the compounds described herein may be for use in therapy, for example, for any of the methods of treatment described herein. Any of the compounds provided herein may be for use in the treatment of a cancer. Any of the compounds provided herein may be for use in the treatment of a glioma.

Any of the modified oligonucleotides described herein may be for use in therapy, for example, for any of the methods of treatment described herein. Any of the modified oligonucleotides provided herein may be for use in the treatment of a cancer. Any of the modified oligonucleotides provided herein may be for use in the treatment of a glioma.

Any of the compounds provided herein may be for use in the preparation of a medicament. Any of the compounds provided herein may be for use in the preparation of a medicament for use in any of the methods of treatment described herein. Any of the compounds provided herein may be for use in the preparation of a medicament for the treatment of a glioma. Any of the compounds provided herein may be for use in the preparation of a medicament for the treatment of a cancer. Any of the compounds provided herein may be for use in the preparation of a medicament for the treatment of a glioma.

Any of the modified oligonucleotides provided herein may be for use in the preparation of a medicament. Any of the modified oligonucleotides provided herein may be for use in the preparation of a medicament for use in any of the methods of treatment described herein. Any of the modified oligonucleotides provided herein may be for use in the preparation of a medicament for the treatment of a cancer. Any of the modified oligonucleotides provided herein may be for use in the preparation of a medicament for the treatment of a glioma.

Any of the pharmaceutical compositions provided herein may be for use in therapy, for example, for any of the methods of treatment described herein. Any of the pharmaceutical compositions provided herein may be for use in the treatment of a cancer. Any of the pharmaceutical compositions provided herein may be for use in the treatment of a glioma.

### Certain Additional Therapies

Cancer treatments often comprise combination therapies. As such, in certain embodiments, the present invention provides methods for treating glioma comprising administering to a subject a compound comprising a modified oligonucleotide, wherein the modified oligonucleotide is complementary to miR-10b, and administering at least one additional therapy that is an anti-cancer therapy. In certain embodiments, an anti-cancer therapy is radiotherapy. In certain embodiments, an anti-cancer therapy is surgical resection of a tumor. In certain embodiments, an anti-cancer therapy is one or more chemotherapeutic agents. In certain embodiments, an anti-cancer therapy is low-intensity, intermediate-frequency alternating electric fields (tumor treating fields, or TTF). In certain embodiments, an anti-cancer therapy is a biological therapy. In certain embodiments, an anti-cancer therapy is a targeted therapy that selected based on one or more genetic abberations in a glioma.

In certain embodiments, anti-cancer therapy comprises a combination of two or more of surgical resection, radiotherapy, chemotherapeutic agents, TTF, and targeted therapy.

In certain embodiments, a subject with glioma is treated with a modified oligonucleotide complementary to miR-10b, surgical resection, radiotherapy, and a chemotherapeutic agent. In certain embodiments, a subject with glioma is treated with a modified oligonucleotide complementary to miR-10b, surgical resection, radiotherapy, TTF, and a chemotherapeutic agent. In certain embodiments, the chemotherapeutic agent is temozolomide. In certain embodiments the chemotherapeutic agent is carmustine.

In certain embodiments, a subject with glioma is treated with a modified oligonucleotide complementary to miR-10b, and surgical resection. In certain embodiments, a subject with glioma is treated with a modified oligonucleotide complementary to miR-10b, radiotherapy, and surgical resection. In certain embodiments, a subject with glioma is treated with a modified oligionucleotides complementary to miR-10b, radiotherapy, surgical resection, and TTF.

In some embodiments, one or more anti-cancer therapies are administered concurrently. In some embodiments, one or more anti-cancer therapies are administered sequentially.

In certain embodiments, the radiotherapy is proton beam therapy. In certain embodiments, the radiotherapy is stereotactic radiosurgery. In certain embodiments, the radiotherapy is intensity-modulated radiotherapy. In certain embodiments, the radiotherapy is 3-D conformal radiation.

In certain embodiments, the TTF is administered using the NovoTTF-100A device (Novocure Ltd, Haifa, Israel). In certain embodiments, the TTF has a frequency of 200 Hz and an intensity of 1-2 V/cm.

In certain embodiments, a chemotherapeutic agent is an alkylating agent. In certain embodiments, a chemotherapeutic agent is an antifolate. In certain embodiments, a chemotherapeutic agent is a growth factor receptor inhibitor. In certain embodiments, a chemotherapeutic agent is an angiogenesis inhibitor. In certain embodiments, a chemotherapeutic agent is a kinase inhibitor. In certain embodiments, a chemotherapeutic agent is an anti-microtubule agent (also known as an alkaloid). In certain embodiments, a chemotherapeutic agent is an alkylating agent. In certain embodiments, a chemotherapeutic agent is an anti-metabolite.

In certain embodiments, a chemotherapeutic agent is selected from 1,3-bis(2-chloroethyl)-1-nitrosourea, busulfan, carboplatin, carmustine, chlorambucil, cisplatin, cyclophosphamide, dacarbazine, daunorubicin, doxorubicin, epirubicin, etoposide, idarubicin, ifosfamide, irinotecan, lomustine, mechlorethamine, melphalan, mitomycin C, mitoxantrone, oxaliplatin, and topotecan.

In certain embodiments, a chemotherapeutic agent is selected from methotrexate, aminopterin, thymidylate synthase, serine hydroxymethyltransferase, folyilpolyglutamyl synthetase, g-glutamyl hydrolase, glycinamide-ribonucleotide transformylase, leucovorin, amino-imidazole-carboxamide-ribonucleotide transformylase, 5-fluorouracil, and a folate transporter.

In certain embodiments, a chemotherapeutic agent is selected from erlotinib and gefitinib.

In certain embodiments, a chemotherapeutic agent is selecte from bevacizumab, thalidomide, carboxyamidotriazole, TNP-470, CM101, IFN-α, platelet factor-4, suramin, SU5416, thrombospondin, a VEGFR antagonist, cartilage-derived angiogenesis inhibitory factor, a matrix metalloproteinase inhibitor, angiostatin, endostatin, 2-methoxyestradiol, tecogalan, tetrathiomolybdate, prolactin, and linomide.

In certain embodiments, a chemotherapeutic agent is selected from BIBW 2992, cetuximab, imatinib, trastuzumab, gefitinib, ranibizumab, pegaptanib, sorafenib, dasatinib, sunitinib, erlotinib, nilotinib, lapatinib, panitumumab, vandetanib, E7080, pazopanib, mubritinib, and fostamatinib.

In certain embodiments, a chemotherapeutic agent is selected from docetaxel and vinblastine.

In certain embodiments, a chemotherapeutic agent is selected from methotrexate and gemcitabine.

Additional suitable anti-cancer therapies include modified oligonucleotides targeted to oncogenic microRNAs other than miR-10b, including but not limited to miR-19, miR-21, and miR-221.

In certain embodiments, the additional therapy is selected to treat or ameliorate a side effect of one or more pharmaceutical compositions of the present invention. Such side effects include, without limitation, nausea, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

Further examples of additional pharmaceutical agents include, but are not limited to, immunoglobulins, including, but not limited to antiemetics; analgesics (e.g., acetaminophen); salicylates; antibiotics; antivirals; antifungal agents; adrenergic modifiers; hormones (e.g., anabolic steroids, androgen, estrogen, calcitonin, progestin, somatostatin, and thyroid hormones); immunomodulators; muscle relaxants; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

### Certain Nucleobase Sequences

The modified oligonucleotides having a nucleoside pattern described herein have a nucleobase sequence that is complementary to miR-10b (SEQ ID NO: 1). In certain embodiments, each nucleobase of the modified oligonucleotide is capable of undergoing base-pairing with a nucleobase at each corresponding position in the nucleobase sequence of miR-10b. In certain embodiments, the nucleobase sequence of a modified oligonucleotide may have one or more mismatched base pairs with respect to the nucleobase sequence of miR-10b or precursor sequence, and remains capable of hybridizing to its target sequence.

In certain embodiments, a modified oligonucleotide consists of a number of linked nucleosides that is equal to the length of miR-10b.

In certain embodiments, the number of linked nucleosides of a modified oligonucleotide is less than the length of miR-10b. A modified oligonucleotide having a number of linked nucleosides that is less than the length of miR-10b, wherein each nucleobase of the modified oligonucleotide is complementary to each nucleobase at a corresponding position of miR-10b, is considered to be a modified oligonucleotide having a nucleobase sequence that is fully complementary (also referred to as 100% complementary) to a region of the miR-10b sequence. For example, a modified oligonucleotide consisting of 19 linked nucleosides, where each nucleobase is complementary to a corresponding position of miR-10b that is 22 nucleobases in length, is fully complementary to a 19-nucleobase region of miR-10b. Such a modified oligonucleotide has 100% complementarity to a 19-nucleobase portion of miR-10b, and is considered to be 100% complementary to miR-10b.

In certain embodiments, a modified oligonucleotide comprises a nucleobase sequence that is complementary to a miR-10b seed sequence, i.e. a modified oligonucleotide comprises a seed-match sequence. In certain embodiments, a seed sequence is a hexamer seed sequence.

In certain embodiments, a modified oligonucleotide has a nucleobase sequence having one mismatch with respect to the nucleobase sequence of miR-10b. In certain embodiments, a modified oligonucleotide has a nucleobase sequence having two mismatches with respect to the nucleobase sequence of miR-10b. In certain such embodiments, a modified oligonucleotide has a nucleobase sequence having no more than two mismatches with respect to the nucleobase sequence of miR-10b. In certain such embodiments, the mismatched nucleobases are contiguous. In certain such embodiments, the mismatched nucleobases are not contiguous.

The nucleobase sequences set forth herein, including but not limited to those found in the examples and in the sequence listing, are independent of any modification to the nucleic acid. As such, nucleic acids defined by a SEQ ID NO may comprise, independently, one or more modifications to one or more sugar moieties, to one or more internucleoside linkages, and/or to one or more nucleobases.

Although the sequence listing accompanying this filing identifies each nucleobase sequence as either “RNA” or “DNA” as required, in practice, those sequences may be modified with a combination of chemical modifications specified herein. One of skill in the art will readily appreciate that in the sequence listing, such designation as “RNA” or “DNA” to describe modified oligonucleotides is somewhat arbitrary. For example, a modified oligonucleotide provided herein comprising a nucleoside comprising a 2′-O-methoxyethyl sugar moiety and a thymine base may be described as a DNA residue in the sequence listing, even through the nucleoside is modified and is not a natural DNA nucleoside.

Accordingly, nucleic acid sequences provided in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” in the sequence listing encompasses any oligonucleotide having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified bases, such as “ATₘₑCGAUCG,” wherein ^{me}C indicates a 5-methylcytosine. Similary, a modified oligonucleotide having the nucleobase sequence “AUCGAUCG” in the sequence listing encompasses any oligonucleotide having such nucleobase sequence, whether modified or unmodified, including, but not limited to, such compounds comprising RNA bases, such as those having sequence “AUCGAUCG” and those having some DNA bases and some RNA bases such as “AUCGATCG” and those having DNA bases such as “ATCGATCG” and oligonucleotides having other modified bases, such as “AT^{me}CGAUCG,” wherein ^{me}C indicates a 5-methylcytosine. In some embodiments, 5-methyluracil (^{m}U) is used to refer to the nucleobase typically referred to as thymine (T).

### Certain Modifications

In certain embodiments, oligonucleotides provided herein may comprise one or more modifications to a nucleobase, sugar, and/or internucleoside linkage, and as such is a modified oligonucleotide. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, a modified oligonucleotide comprises one or more modified nucleosides.

In certain embodiments, a modified nucleoside is a sugar-modified nucleoside. In certain such embodiments, the sugar-modified nucleosides may further comprise a natural or modified heterocyclic base moiety and/or may be connected to another nucleoside through a natural or modified internucleoside linkage and/or may include further modifications independent from the sugar modification. In certain embodiments, a sugar modified nucleoside is a 2′-modified nucleoside, wherein the sugar ring is modified at the 2′ carbon from natural ribose or 2′-deoxy-ribose.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

Nucleosides comprising such bicyclic sugar moieties are referred to as bicyclic nucleosides or BNAs. In certain embodiments, bicyclic nucleosides include, but are not limited to, (A) α-L-methyleneoxy (4′-CH₂—O-2′) BNA; (B) β-D-methyleneoxy (4′-CH₂—O-2′) BNA; (C) ethyleneoxy (4′-(CH₂)₂—O-2′) BNA; (D) aminooxy (4′-CH₂—O—N(R)-2′) BNA; (E) oxyamino (4′-CH₂—N(R)—O-2′) BNA; (F) methyl(methyleneoxy) (4′-CH(CH₃)—O-2′) BNA (also referred to as constrained ethyl or cEt); (G) methylene-thio (4′-CH₂—S-2′) BNA; (H) methylene-amino (4′-CH₂—N(R)-2′) BNA; (I) methyl carbocyclic (4′-CH₂—CH(CH₃)-2′) BNA; (J) c-MOE (4′-CH(CH₂—OMe)-O-2′) BNA and (K) propylene carbocyclic (4′-(CH₂)₃-2′) BNA as depicted below.

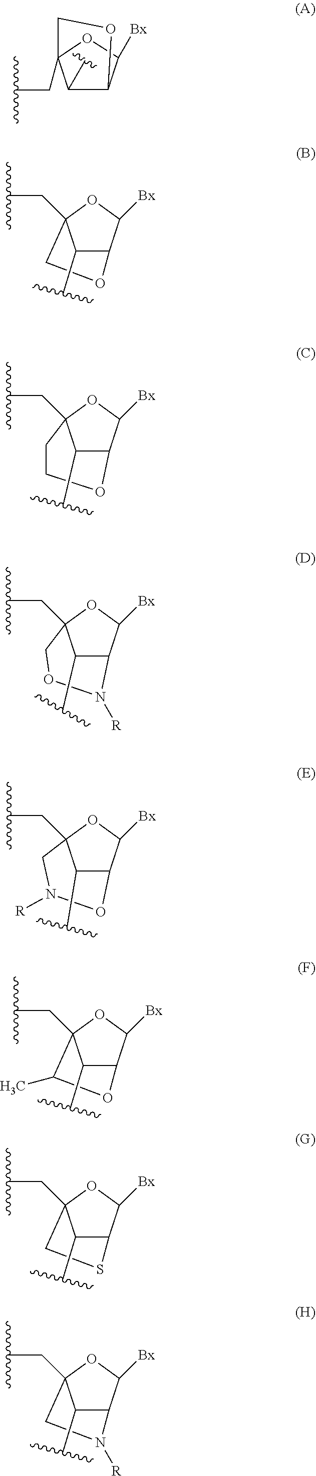

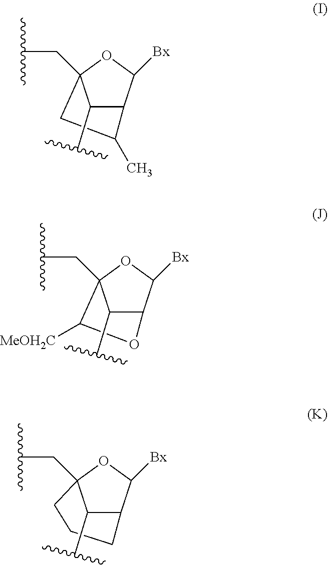

wherein Bx is a nucleobase moiety and R is, independently, H, a protecting group, or C₁-C₁₂ alkyl.

In certain embodiments, a 2′-modified nucleoside comprises a 2′-substituent group selected from F, OCF₃, O—CH₃ (also referred to as “2′-OMe”), OCH₂CH₂OCH₃ (also referred to as “2′-O-methoxyethyl” or “2′-MOE”), 2′-O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-modified nucleoside comprises a 2′-substituent group selected from F, O—CH₃, and OCH₂CH₂OCH₃.

In certain embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4-O replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O CH₂CH₂OCH₃, and 2′-F.

In certain embodiments, a modified oligonucleotide comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In certain embodiments, each internucleoside linkage of a modified oligonucleotide is a phosphorothioate internucleoside linkage.

In certain embodiments, a modified oligonucleotide comprises one or more modified nucleobases.

In certain embodiments, a modified nucleobase is selected from 5-hydroxymethyl cytosine, 7-deazaguanine and 7-deazaadenine. In certain embodiments, a modified nucleobase is selected from 7-deaza-adenine, 7-deazaguanosine, 2-aminopyridine and 2-pyridone. In certain embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In certain embodiments, a modified nucleobase comprises a polycyclic heterocycle. In certain embodiments, a modified nucleobase comprises a tricyclic heterocycle. In certain embodiments, a modified nucleobase comprises a phenoxazine derivative. In certain embodiments, the phenoxazine can be further modified to form a nucleobase known in the art as a G-clamp.

In certain embodiments, a modified oligonucleotide is conjugated to one or more moieties which enhance the activity, cellular distribution or cellular uptake of the resulting antisense oligonucleotides. In certain such embodiments, the moiety is a cholesterol moiety. In certain embodiments, the moiety is a lipid moiety. Additional moieties for conjugation include carbohydrates, peptides, antibodies or antibody fragments, phospholipids, biotin, phenazine, folate, phenanthridine, anthraquinone, acridine, fluoresceins, rhodamines, coumarins, and dyes. In certain embodiments, the carbohydrate moiety is N-acetyl-D-galactosamine (GalNac). In certain embodiments, a conjugate group is attached directly to an oligonucleotide. In certain embodiments, a conjugate group is attached to a modified oligonucleotide by a linking moiety selected from amino, azido, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, azido, carboxy, benzyl, phenyl, nitro, thiol, thioalkoxy, halogen, alkyl, aryl, alkenyl and alkynyl.

In certain such embodiments, the compound comprises a modified oligonucleotide having one or more stabilizing groups that are attached to one or both termini of a modified oligonucleotide to enhance properties such as, for example, nuclease stability. Included in stabilizing groups are cap structures. These terminal modifications protect a modified oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

### Certain Pharmaceutical Compositions

Provided herein are pharmaceutical compositions comprising a compound provided herein, and a pharmaceutically acceptable diluent. In certain embodiments, the pharmaceutically acceptable diluent is an aqueous solution. In certain embodiments, the aqueous solution is a saline solution. As used herein, pharmaceutically acceptable diluents are understood to be sterile diluents. Suitable administration routes include, without limitation, intratumoral, intracranial, intrathecal, intravenous and subcutaneous administration. In certain embodiments, intracranial administration comprises intracranial implantation of a device comprising a chemotherapeutic agent and biodegradable copolymer to control the release of a pharmaceutical composition provided herein. In certain embodiments, the implantable device comprises carmustine. In certain embodiments, the implantable device is a Gliadil® wafer.

In certain embodiments, a pharmaceutical composition is administered in the form of a dosage unit. For example, in certain embodiments, a dosage unit is in the form of a tablet, capsule, implantable device, or a bolus injection.

In certain embodiments, a pharmaceutical agent is a modified oligonucleotide which has been prepared in a suitable diluent, adjusted to pH 7.0-9.0 with acid or base during preparation, and then lyophilized under sterile conditions. The lyophilized modified oligonucleotide is subsequently reconstituted with a suitable diluent, e.g., aqueous solution, such as water or physiologically compatible buffers such as saline solution, Hanks's solution, or Ringer's solution. The reconstituted product is administered as a subcutaneous injection or as an intravenous infusion. The lyophilized drug product may be packaged in a 2 mL Type I, clear glass vial (ammonium sulfate-treated), stoppered with a bromobutyl rubber closure and sealed with an aluminum overseal.

In certain embodiments, the pharmaceutical compositions provided herein may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents.

In some embodiments, the pharmaceutical compositions provided herein may contain additional materials useful in physically formulating various dosage forms of the compositions of the present invention, such as dyes, flavoring agents, preservatives, antioxidants, opacifiers, thickening agents and stabilizers; such additional materials also include, but are not limited to, excipients such as alcohol, polyethylene glycols, gelatin, lactose, amylase, magnesium stearate, talc, silicic acid, viscous paraffin, hydroxymethylcellulose and polyvinylpyrrolidone. In various embodiments, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions. Lipid moieties have been used in nucleic acid therapies in a variety of methods. In one method, the nucleic acid is introduced into preformed liposomes or lipoplexes made of mixtures of cationic lipids and neutral lipids. In another method, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, a pharmaceutical composition provided herein comprises a polyamine compound or a lipid moiety complexed with a nucleic acid. In certain embodiments, such preparations comprise one or more compounds each individually having a structure defined by formula (Z) or a pharmaceutically acceptable salt thereof,

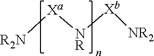

wherein each X^{a} and X^{b}, for each occurrence, is independently C₁₋₆ alkylene; n is 0, 1, 2, 3, 4, or 5; each R is independently H, wherein at least n+2 of the R moieties in at least about 80% of the molecules of the compound of formula (Z) in the preparation are not H; m is 1, 2, 3 or 4; Y is O, NR², or S; R¹ is alkyl, alkenyl, or alkynyl; each of which is optionally substituted with one or more substituents; and R² is H, alkyl, alkenyl, or alkynyl; each of which is optionally substituted each of which is optionally substituted with one or more substituents; provided that, if n=0, then at least n+3 of the R moieties are not H. Such preparations are described in PCT publication WO/2008/042973, which is herein incorporated by reference in its entirety for the disclosure of lipid preparations. Certain additional preparations are described in Akinc et al., Nature Biotechnology 26, 561-569 (1 May 2008), which is herein incorporated by reference in its entirety for the disclosure of lipid preparations.

In certain embodiments, a pharmaceutical composition provided herein is prepared using known techniques, including, but not limited to mixing, dissolving, granulating, dragee-making, levigating, emulsifying, encapsulating, entrapping or tableting processes.

In certain embodiments, a pharmaceutical composition provided herein is a solid (e.g., a powder, tablet, and/or capsule). In certain of such embodiments, a solid pharmaceutical composition comprising one or more oligonucleotides is prepared using ingredients known in the art, including, but not limited to, starches, sugars, diluents, granulating agents, lubricants, binders, and disintegrating agents.

In certain embodiments, a pharmaceutical composition provided herein is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Certain pharmaceutical compositions for injection are presented in unit dosage form, e.g., in ampoules or in multi-dose containers.

In certain embodiments, a pharmaceutical composition provided herein comprises a modified oligonucleotide in a therapeutically effective amount. In certain embodiments, the therapeutically effective amount is sufficient to prevent, alleviate or ameliorate symptoms of a disease or to prolong the survival of the subject being treated.

In certain embodiments, one or more modified oligonucleotides provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

Additional administration routes include, but are not limited to, oral, rectal, transmucosal, intestinal, enteral, topical, suppository, through inhalation, intrathecal, intracardiac, intraventricular, intraperitoneal, intranasal, intraocular, intratumoral, intramuscular, and intramedullary administration. In certain embodiments, pharmaceutical intrathecals are administered to achieve local rather than systemic exposures. For example, pharmaceutical compositions may be injected directly in the area of desired effect.

### Certain Kits

The present invention also provides kits. In some embodiments, the kits comprise one or more compounds comprising a modified oligonucleotide disclosed herein. In some embodiments, the kits may be used for administration of the compound to a subject.

In certain embodiments, the kit comprises a pharmaceutical composition ready for administration. In certain embodiments, the pharmaceutical composition is present within a vial. In certain embodiments, the pharmaceutical composition is present within an implantable device. A plurality of vials or implantable devices, such as 10, can be present in, for example, dispensing packs. In some embodiments, the vial is manufactured so as to be accessible with a syringe. The kit can also contain instructions for using the compounds.

In some embodiments, the kit comprises a pharmaceutical composition present in a pre-filled syringe (such as a single-dose syringes with, for example, a 27 gauge, ½ inch needle with a needle guard), rather than in a vial. A plurality of pre-filled syringes, such as 10, can be present in, for example, dispensing packs. The kit can also contain instructions for administering the compounds comprising a modified oligonucleotide disclosed herein.

In some embodiments, the kit comprised a modified oligonucleotide provided herein as a lyophilized drug product, and a pharmaceutically acceptable diluent. In preparation for administration to a subject, the lyophilized drug product is reconstituted in the pharmaceutically acceptable diluent.

In some embodiments, in addition to compounds comprising a modified oligonucleotide disclosed herein, the kit can further comprise one or more of the following: syringe, alcohol swab, cotton ball, and/or gauze pad.

### Certain Experimental Models

In certain embodiments, the present invention provides methods of using and/or testing modified oligonucleotides of the present invention in an experimental model. Those having skill in the art are able to select and modify the protocols for such experimental models to evaluate a pharmaceutical agent of the invention.

Generally, modified oligonucleotides are first tested in cultured cells. Suitable cell types include those that are related to the cell type to which delivery of a modified oligonucleotide is desired in vivo. For example, suitable cell types for the study of the methods described herein include primary or cultured cells.

In certain embodiments, the extent to which a modified oligonucleotide interferes with the activity of miR-10b is assessed in cultured cells. In certain embodiments, inhibition of microRNA activity may be assessed by measuring the levels of the microRNA. Alternatively, the level of a predicted or validated microRNA-regulated transcript may be measured. An inhibition of microRNA activity may result in the increase in the miR-10b-regulated transcript, and/or the protein encoded by miR-10b-regulated transcript. Further, in certain embodiments, certain phenotypic outcomes may be measured.

Several animal models are available to the skilled artisan for the study of miR-10b in models of human disease. For example, inhibitors of miR-10b may be studied in models of cancer, such as orthotopic xenograft models, toxin-induced cancer models, or genetically-induced cancer models. In such cancer models, the studies may be performed to evaluate the effects of inhibitors of miR-10b on tumor size, tumor number, overall survival and/or progression-free survival. Suitable animal models include, without limitation, a glioma-derived xenograft model and a glioma-derived orthotopic model. The xenograft and orthotopic models may be established with cultured glioma cells, or with glioma cells isolated from a surgical sample.

### Certain Quantitation Assays

In certain embodiments, microRNA levels are quantitated in cells or tissues in vitro or in vivo. In certain embodiments, changes in microRNA levels are measured by microarray analysis. In certain embodiments, changes in microRNA levels are measured by one of several commercially available PCR assays, such as the TaqMan® MicroRNA Assay (Applied Biosystems).

Modulation of microRNA activity with an anti-miR or microRNA mimic may be assessed by microarray profiling of mRNAs. The sequences of the mRNAs that are modulated (either increased or decreased) by the anti-miR or microRNA mimic are searched for microRNA seed sequences, to compare modulation of mRNAs that are targets of the microRNA to modulation of mRNAs that are not targets of the microRNA. In this manner, the interaction of the anti-miR with its target microRNA, or a microRNA mimic with its targets, can be evaluated. In the case of an anti-miR, mRNAs whose expression levels are increased are screened for the mRNA sequences that comprise a seed match to the microRNA to which the anti-miR is complementary.

Modulation of microRNA activity with an anti-miR compound may be assessed by measuring the level of a messenger RNA target of the microRNA, either by measuring the level of the messenger RNA itself, or the protein transcribed therefrom. Antisense inhibition of a microRNA generally results in the increase in the level of messenger RNA and/or protein of the messenger RNA target of the microRNA, i.e., anti-miR treatment results in de-repression of one or more target messenger RNAs.

### EXAMPLES

The following examples are presented in order to more fully illustrate some embodiments of the invention. They should, in no way be construed, however, as limiting the broad scope of the invention. Those of ordinary skill in the art will readily adopt the underlying principles of this discovery to design various compounds without departing from the spirit of the current invention.

### Example 1: The Role of miR-10b in Glioma

Previous studies using a research tool anti-miR-10b modified oligonucleotide (MO) in an mouse model of glioma demonstrated that inhibition of miR-10b significantly reduced tumor growth. While the compound tested in this model demonstrated efficacy, no data was provided related to safety of the compound, or its suitability for use in human subjects with glioma. In general, due to a lack of testing for safety as pharmaceutical agents, research tool compounds are unlikely to be suitable for use in human subjects having glioma. In view of this, a screen was performed to identify anti-miR-10b compounds that are sufficiently efficacious, convenient to administer, and safe for administration to human subjects having glioma.

Approximately 215 anti-miR-10b compounds were designed, having varying lengths and chemical composition. The length of the compounds ranged from 9 to 23 linked nucleosides, and the compounds varied in the number, type, and placement of chemical modifications. As potency and safety cannot be predicted based on a compound's chemical structure, compounds were evaluated both in vitro and in vivo for characteristics including potency, efficacy, pharmacokinetic behavior, safety, and metabolic stability, in a series of assays designed to eliminate compounds with unfavorable properties. In certain assays, the tool anti-miR-10b compound was used as a benchmark to which the other anti-miR-10b compounds are compared. Each of the approximately 215 compounds was first tested in several in vitro assays (e.g. potency, toxicology), to identify a smaller set of compounds suitable for further testing in more complex in vivo assays (e.g. pharmacokinetic profile, efficacy, toxicology).

Also tested in each of these assays was a research tool compound, RG348124, 5′-CACAAATTCGGTTCTACAGGGTA-3′ (SEQ ID NO: 3), where each nucleoside comprises a 2′-O-methoxyethyl sugar moiety, each C is a 5-methylcytosine, and each internucleoside linkage is a phosphorothioate internucleoside linkage.

As a first step in the screening cascade, compounds were tested for potential toxicity using a biochemical fluorescent binding assay (FBA). The FBA was performed by incubating a fluorescent dye with each compound, and immediately measuring fluorescence. Highly fluorescent compounds have the potential to produce toxicity in vivo and were not included in further testing.

In vitro potency was evaluated using a luciferase reporter assay. A luciferase reporter plasmid for miR-10b was designed, with a fully complementary miR-10b binding site in the 3′-UTR of the luciferase gene. A stable Hela cell line expressing this luciferase construct was generated. Cells were transfected to introduce miR-10b, which represses the expression of luciferase from the reporter construct. Subsequent transfection of the cells with active anti-miR-10b compound inhibits that activity of miR-10b, and increases luciferase mRNA expression resulting in an increased luminescence signal. Cells were treated with anti-miR-10b compound at concentrations of 1 nM, 10 nM, and 100 nM. Compounds of longer lengths were identified as suitably active if their EC₅₀ (concentration that yields a half-maximal response) was less than or equal to 5 nM. As shorter compounds, such as 9-mers, are typically not maximally active in the same assay conditions used for longer compounds, shorter compounds were selected based on maximum inhibition relative to appropriate control compounds. In this way, compounds that are diverse in both length and chemical composition were included in further testing.

Based on data from the luciferase assay and FBA, and consideration of chemical diversity, certain compounds were chosen for further testing in a liver slice assay. The liver slice assay, designed to identify compounds with the potential to cause toxicity, was performed by incubating individual compounds with a slice of tissue from a core liver sample isolated from rat liver. Following a 24-hour incubation, RNA is extracted from the liver slice, and the expression levels of several pro-inflammatory genes, including IFIT, are measured. A log 2 transformation of the fold change (Log 2-FC) relative to PBS treatment was performed. An induction in pro-inflammatory gene expression indicates a potential for pro-inflammatory effects (i.e., toxicity) in vivo, and thus these compounds are excluded from further testing.

Metabolic stability was evaluated by incubating each anti-miR-10b compound in a mouse liver or brain lysate. After 24 hours, the percentage of intact compound remaining is calculated. Compounds that are not stable following a 24-hour incubation are potentially not stable in vivo.

As oligonucleotides are typically administered via subcutaneous injection, compounds of lower viscosity are preferred. Generally, a viscosity of less than 40 cP at a concentration of 150 mg/ml was considered acceptable for a formulation intended for administration by subcutaneous injection. Higher viscosities may be acceptable for compounds administered by other methods, such as by intravenous injection an implantable device.

Based on these assays, certain compounds were selected for further testing in assays for caspase activity, cell viability, metabolic stability, viscosity and toxicity in an acute setting. These compounds, shown in Table 1, are candidate therapeutic agents for the treatment of glioma.

TABLE 1
anti-miR-10b Compounds

| Compound | | | SEQ |
|---|---|---|---|
| # | Nuclebase Sequence and Chemistry (5′ to 3′) | Length | ID NO |
| RG5452 | C_{K}A_{K}^{m}C_{E}A_{K}A_{E}A_{E}U_{K}T_{E}^{m}C_{E}G_{K}G_{E}T_{E}U_{K}^{m}C_{E}T_{E}A_{K}^{m}C_{E}A_{E}G_{E}G_{E}G_{E}T_{E}A_{E} | 23 | 15 |
| RG5454 | C_{K}A_{K}^{m}C_{E}A_{E}A_{K}A_{E}T_{E}U_{K}^{m}C_{E}G_{K}G_{E}T_{E}T_{E}^{m}C_{E}T_{E}A_{E}^{m}C_{E}A_{E}G_{E}G_{E}G_{E}T_{E}A_{E} | 23 | 13 |
| RG5455 | ^{m}C_{E}A_{E}^{m}C_{E}A_{E}A_{E}A_{E}U_{K}T_{E}^{m}C_{E}G_{K}G_{E}T_{E}U_{K}^{m}C_{E}T_{E}A_{K}^{m}C_{E}A_{E}G_{E}G_{E}G_{E}T_{E}A_{E} | 23 | 15 |
| RG5461 | C_{K}A_{K}A_{E}A_{E}U_{K}T_{E}^{m}C_{E}G_{K}G_{E}T_{E}U_{K}^{m}C_{E}T_{E}A_{K}^{m}C_{E}A_{E}G_{E}G_{E}G_{E}T_{E}A_{E} | 21 | 11 |
| RG5470 | A_{K}A_{E}U_{K}T_{E}^{m}C_{E}G_{K}G_{E}T_{E}U_{K}^{m}C_{E}T_{E}A_{K}^{m}C_{E}A_{E}G_{E}G_{E}G_{E}T_{E}A_{E} | 19 | 7 |
| RG5476 | U_{K}U_{K}^{m}C_{E}G_{K}G_{E}T_{E}U_{K}^{m}C_{E}T_{E}A_{K}^{m}C_{E}A_{E}G_{E}G_{E}G_{E}T_{E}A_{E} | 17 | 19 |
| RG5552 | C_{K}A_{K}C_{K}AA_{K}A_{K}U_{K}TC_{K}G_{E}G_{E}T_{E}T_{E}^{m}C_{E}T_{E}A_{E}^{m}C_{E}A_{E}G_{E}G_{E}G_{E}T_{E}A_{E} | 23 | 14 |
| RG5553 | C_{K}A_{K}C_{K}A_{K}AA_{K}U_{K}U_{K}C_{K}G_{E}G_{E}T_{E}T_{E}^{m}C_{E}T_{E}A_{E}^{m}C_{E}A_{E}G_{E}G_{E}G_{E}T_{E}A_{E} | 23 | 16 |
| RG5556 | ^{m}C_{E}A_{E}A_{E}A_{E}T_{E}T_{E}^{m}C_{E}G_{E}G_{E}T_{E}U_{K}CTA_{K}CAG_{K}GGU_{K}A_{K} | 21 | 8 |
| RG5577 | C_{K}A_{K}AA_{K}TU_{K}CG_{K}GT_{E}T_{E}^{m}C_{E}T_{E}A_{E}^{m}C_{E}A_{E}G_{E}G_{E}G_{E}T_{E}A_{E} | 21 | 9 |
| RG5578 | C_{K}A_{K}AA_{K}U_{K}TC_{K}G_{K}GT_{E}T_{E}^{m}C_{E}T_{E}A_{E}^{m}C_{E}A_{E}G_{E}G_{E}G_{E}T_{E}A_{E} | 21 | 10 |
| RG5579 | C_{K}A_{K}A_{K}AU_{K}U_{K}C_{K}GG_{K}T_{E}T_{E}^{m}C_{E}T_{E}A_{E}^{m}C_{E}A_{E}G_{E}G_{E}G_{E}T_{E}A_{E} | 21 | 12 |
| RG5580 | C_{K}A_{K}A_{K}A_{K}TU_{K}C_{K}G_{K}G_{K}T_{E}T_{E}^{m}C_{E}T_{E}A_{E}^{m}C_{E}A_{E}G_{E}G_{E}G_{E}T_{E}A_{E} | 21 | 9 |
| RG5606 | A_{K}A_{K}U_{K}TC_{K}G_{K}G_{K}TU_{K}^{m}C_{E}T_{E}A_{E}^{m}C_{E}A_{E}G_{E}G_{E}G_{E}T_{E}A_{E} | 19 | 7 |
| RG5634 | A_{E}A_{K}T_{E}T_{E}C_{K}G_{E}G_{E}U_{K}U_{M}C_{M}U_{K}A_{M}C_{M}A_{K}G_{M}G_{M}G_{K}U_{K}A_{K} | 19 | 5 |
| RG5646 | A_{E}A_{E}T_{E}T_{E}C_{K}GG_{K}TU_{K}C_{M}U_{K}A_{M}C_{K}A_{M}G_{K}G_{M}G_{K}U_{K}A_{E} | 19 | 4 |
| RG5648 | A_{E}A_{E}T_{E}T_{E}^{m}C_{E}G_{E}G_{E}U_{K}U_{K}C_{M}U_{K}A_{K}C_{M}A_{K}G_{K}G_{M}G_{K}U_{K}A_{E} | 19 | 5 |
| RG5650 | A_{E}A_{E}U_{K}T_{E}^{m}C_{E}G_{K}G_{E}T_{E}ToCoU_{K}A_{K}C_{M}AfG_{F}G_{F}G_{M}U_{K}A_{K} | 19 | 6 |
| RG5655 | U_{K}A_{KM}A_{F}G_{F}G_{F}G_{M}U_{K}A_{K}^{o}A^{o}A^{o}U_{K}A_{K}C_{M}A_{F}G_{F}G_{F}G_{M}U_{K}A_{K} | 20 | 18 |
| RG5656 | C_{K}U_{K}A_{M}C_{F}A_{F}G_{M}G_{F}G_{M}U_{K}A_{K}^{o}A^{o}A^{o}C_{K}U_{K}A_{M}C_{F}A_{F}G_{M}G_{F}G_{M}Y_{K}A_{K} | 22 | 17 |
| RG5658 | U_{K}A_{K}C_{M}A_{F}G_{F}G_{F}G_{M}U_{K}A_{K} | 9 | |

In the compounds in Table 1, nucleosides followed by subscript “E” are 2′-O-methoxyethyl nucleosides, nucleosides followed by subscript “M” are 2′-O-methyl nucleosides, nucleosides followed by subscript “F” are 2′-fluoro nucleosides, nucleosides followed by subscript “K” are S-cEt nucleosides, “U” is a non-methylated uracil, “^{m}C” is a 5-methyluracil, “^{m}C” is a 5-methylcytosine, “C” is a non-methylated cytosine, “A” is an adenine, “G” is a guanine; a superscript “0” indicates a phosphodiester linkage and each other internucleoside linkage is a phosphorothioate linkage.

### Example 2: Anti-miR-10b Compound Testing in Further Assays

In evaluating candidate therapeutic agents for the treatment of cancer, relevant cellular assays include a cell viability and an apoptosis induction assay. For these assays, glioblastoma-derived cell lines were used.

For the cell viability assay, approximately 8,000 cells were plated into each well of a 96-well plate. The following day, cells were transfected with anti-miR-10b compound at doses of 2, 4, 8, 16, 31, 63, 125, 250, and 500 nM, using RNAiMAX™ as the transfection reagent. After 72 hours, cell viability was determined using the CellTiter-Glo® Luminescent Cell Viability Assay. An IC₅₀ was calculated for each compound. The assay was performed using both LN229, U87, MCF7, and HCN2 cells.

Caspase activity was used as an indicator of the induction of apoptosis. Approximately 8,000 cells were plated into each well of a 96-well plate. The following day, cells were transfected with anti-miR-10b compound using RNAiMAX™. After 48 hours, caspase 3/7 activity was determined using the Caspase-Glo 3/7 Assay System (Promega). An EC₅₀ was calculated for each compound. LN229 cells were used for this assay.

Based on these functional assays, three compounds were selected based on potency. Of the compounds of longer lengths, RG5579 and RG5461 were the highest ranked according to IC₅₀ in the viability assay; of the compounds of shorter lengths, RG5658 was the highest ranked according to IC₅₀ in the viability assay. The results from the luciferase, viability and caspase assays are shown in Table 2. The research tool compound is included as a benchmark for activity in the various assays.

TABLE 2
In Vitro Activity of Lead Anti-miR-10b Compounds

| Assay | RG384124 | RG5579 | RG5461 | RG5658 |
|---|---|---|---|---|
| Luciferase (avg fold change) | 2.8 | 12.6 | 12.4 | 1.8 |
| 1 nM | | | | |
| Luciferase (avg fold change) | 29.5 | 39.1 | 49.8 | 3.7 |
| 10 nM | | | | |
| Luciferase (avg fold change) | 55.6 | 27.8 | 35.1 | 4.7 |
| 100 nM | | | | |
| LN229 viability assay IC₅₀ | 43 nM | 12.2 nM | 13.2 nM | 90.1 nM |
| U87 viability assay IC₅₀ | 31.3 nM | 24.8 nM | 10.1 nM | 45.6 nM |
| LN229 caspase 3 activation | 85.1 nM | 13.1 nM | 21.1 nM | 589.7 nM |
| assay IC₅₀ | | | | |
| MCF7 viability assay IC₅₀ | 80.5 nM | 65.5 nM | 96.8 nM | 928.6 nM |
| HCN2 viability assay IC₅₀ | 44.4 nM | 85.9 nM | 134 nM | N.D. |

The compounds with the greatest activity in the functional assays are also evaluated for potential systemic toxicity, using an in vivo assay in normal, Sv129 mice. A single, subcutaneous dose of 300 mg/kg of anti-miR-10b was administered. Included as control treatments were PBS, and two anti-miRs not related to miR-17, one known to be pro-inflammatory (positive control) and one that is not pro-inflammatory (negative control). Four days later, mice were sacrificed. Kidney and liver tissue was isolated for RNA extraction. The level of two genes known to be induced during an inflammatory response, IFIT and OASL2, were measured and normalized to mouse GAPDH. A log 2 transformation of the fold change (Log 2-FC) relative to PBS treatment was performed.

Based on the assays described in Example 1, the viability and caspase assays, and the systemic toxicity assay, three compounds, RG5579, RG5461, and RG5658 were identified as having suitable profiles with regard to potency and lack of potential toxicity in in vitro assays.

### Example 3: In Vitro Efficacy in Combination with Temozolomide

To evaluate the effect of miR-10b inhibition on the activity of temozolomide (TMZ), LN229 cells were treated with anti-miR-10b compound and TMZ. RG5579 and RG5461 were selected for testing in this assay.

Approximately 8,000 cells were plated into each well of a 96-well plate. The following day, cells were treated with anti-miR-10b compound at concentrations of 0, 5, 10 or 20 nM of RG5579 or RG5461, in addition to TMZ at concentrations ranging from 0 to 200 uM. After 72 hours, cell viability was determined using the CellTiter-Glo® Luminescent Cell Viability Assay. An IC₅₀ for TMZ at each concentration of anti-miR-10b concentration was calculated and is shown in Table 3.

TABLE 3
IC₅₀ for TMZ in the presence of anti-miR-10b

| Anti-miR-10 | Concentration of | |
|---|---|---|
| Compound | anti-miR-10b (nM) | TMZ IC₅₀ (uM) |
| RG5579 | 0 | 10.55 |
| | 5 | 11.4 |
| | 10 | 10.45 |
| | 20 | 1.029 |
| RG5461 | 0 | 10.5 |
| | 5 | 10.85 |
| | 10 | 9.335 |
| | 20 | 4.27 |

As shown in Table 3, RG5579 and RG5461 each decreased the IC₅₀ of TMZ in the LN229 viability assay, and thus significantly enhanced the potency of TMZ in vitro.

### Example 3: In Vivo Testing in GBM Models

To determine the effects of modified oligonucleotides targeted to miRNAs on tumor growth, anti-miR-10b compounds were was evaluated in mouse model of gliomas, for effects on tumor size, tumor growth, and survival.

Subcutaneous Xenograft Model:

Human gliomablastoma-derived cells growing in culture are trypsinzed, counted, and resuspended in a 1:1 mixture of media:growth factor reduced Matrigel. Approximately 10⁶ cells, in a volume of 100 ul, are injected subcutaneously into the flank of nude mice. Ten days following subcutaneous tumor implantation, tumor size is measured using calipers, and mice are randomized into treatment groups. Intratumoral (e.g., 5 ug/30 mm³ tumor), subcutaneous (e.g., 100 mg/kg) or intravenous (e.g., 80 mg/kg) dosing of anti-miR-10b compound begins post-implantation. Tumor size is measured three to five days per week. Final tumor size and weight are measured at the end of the study.

Orthotopic Model:

Human gliomablastoma-derived cells growing in culture are trypsinzed, counted, and resuspended PBS. The cells express a fluorescent marker and luciferase insertion that enables monitoring of tumor growth and size via an in vivo imaging system. Approximately 5×10⁵ cells, in a volume of 5 ul, are injected into the brain of nude mice. Following intracranial tumor implantation, tumor burden is measured using the IVIS Spectrum In Vivo Imaging System (PerkinElmer), and mice are randomized into treatment groups. Intratumoral (e.g., 0.1-500 ug/tumor), subcutaneous (e.g., 100 mg/kg) or intravenous (e.g., 80 mg/kg) dosing of anti-miR-10b compound begins post-implantation. Tumor burden is measured weekly using the imaging system. Final tumor size and weight are measured at the end of the study.

Three candidate therapeutic agents, RG5579, RG5461, and RG5658 were tested in the subcutaneous and orthotopic glioma models. RG5580, the third most potent compound in the in vitro viability assay, was also tested. The studies were designed to evaluate subcutaneous administration vs. intratumoral administration in subcutaneous in orthotopic models; efficacy of the anti-miR-10b compound alone and in combination with TMZ; and efficacy of a single injection of anti-miR-10b compound vs multiple injections. The research tool compound RG384124 was also tested.

### RG5579 and RG5580 in an Orthotopic Glioblastoma Model

RG5658 and RG5461 were tested in an orthotopic model of GBM established with LN229 cells.

Human gliomablastoma-derived LN229 cells growing in culture were trypsinized, counted, and resuspended PBS. The cells express a fluorescent marker that enables monitoring of tumor growth and size during treatment. Approximately 5×10⁵ cells, in a volume of 5 ul, were injected into the brain of nude mice on Day 0. Mice were randomized into the following treatment groups, with 8 mice per group: (1) PBS; (2) RG5579; (3) RG5580; and (4) negative control. On Day 29, mice were given an intratumoral injection of either PBS or 50 ug of anti-miR-10b compound or negative control. On Day 48, mice were given PBS or 30 ug of anti-miR-10b or negative control. Survival was monitored, and overall median survival was determined.

As shown in Table 4, treatment with RG5579 improved overall median survival by 14%, relative to PBS treatment.

TABLE 4
Anti-miR-10b Improves Median Survival in GBM Model

| | Median | |
|---|---|---|
| | Survival in | % increase |
| Treatment | Days | (relative to PBS) |
| PBS | 59.5 | — |
| Neg. control | 58 | — |
| RG5580 | 57 | — |
| RG5579 | 68 | 14% |

### RG5461 and RG5658 in an Orthotopic Glioblastoma Model

RG5658 and RG5461 were tested in an orthotopic model of GBM established with LN229 cells. Treatments comprised anti-miR-10b compound alone or in combination with TMZ.

Initial testing in subcutaneous and orthotopic models revealed that a single dose of RG5668 or RG5461, administered intratumorally, consistently delayed tumor growth, but did not significantly improve overall survival.

Further testing was performed to evaluate the effects of RG5658 and RG5461 treatment in combination with TMZ. Human gliomablastoma-derived LN229 cells growing in culture were trypsinzed, counted, and resuspended PBS. The cells express a fluorescent marker that enables monitoring of tumor growth and size during treatment. Approximately 5×10⁵ cells, in a volume of 5 ul, were injected into the brain of nude mice on Day 0. Mice were randomized into the following treatment groups, with 8 mice per group: (1) PBS; (2) RG5461; (3) RG5658; (4) RG5461+TMZ; (5) RG5658+TMZ; and (6) PBS+TMZ. On Day 21, a single dose of anti-miR-10b compound was administered intratumorally at a dose of 20 ug for RG5658 or 50 ug for RG5461. TMZ was administered daily on each of Days 35-41. Survival was monitored, and overall median survival was determined. As shown in Table 5, the combination of anti-miR-10b compound and TMZ improved median survival, relative to TMZ alone or to anti-miR-10b compound alone.

TABLE 5
Anti-miR-10b + TMZ Improves Median Survival in GBM Model

| | Median | |
|---|---|---|
| | Survival in | % increase |
| Treatment | Days | (relative to PBS) |
| PBS | 55.5 | — |
| RG5461 | 59 | — |
| RG5658 | 56 | — |
| TMZ | 67.5 | 22% |
| RG5461 + TMZ | 88 | 59% |
| RG5668 + TMZ | 95 | 71% |

### RG5579 in an Orthotopic Glioblastoma Model

RG5579 was tested in an orthotopic model of GBM established with LN229 cells.

Human gliomablastoma-derived LN229 cells growing in culture were trypsinized, counted, and resuspended PBS. The cells express a fluorescent marker that enables monitoring of tumor growth and size during treatment. Approximately 5×10⁵ cells, in a volume of 5 ul, were injected into the brain of nude mice on Day 0. Mice were randomized into the following treatment groups, with 8 mice per group: (1) PBS; (2) RG5579; (3) PBS+TMZ; and (4) RG5579+TMZ. On Day 21, mice were given an intratumoral injection of either PBS or 40 ug of RG5579. On Days 35-41, TMZ was administered daily. Survival was monitored, and overall median survival was determined.

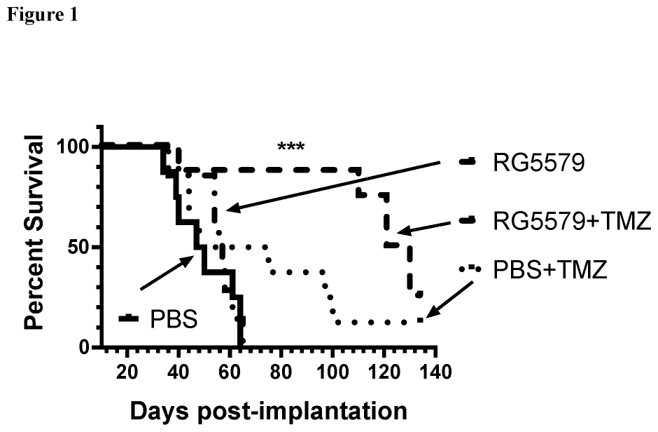

The percent survival curve is shown in FIG. 1, and percent increases in overall survival are shown in Table 6. These results demonstrate that RG5579 as a single agent treatment increases the median survival of mice, relative to PBS treatment. Treatment with both RG5579 and TMZ resulted in an even greater increase in median survival.

TABLE 6
Median Overall Survival in GBM Mouse Model
Median Overall Survival

| | | % increase |
|---|---|---|
| | Days | (relative to PBS) |
| PBS | 48.5 | — |
| RG5579 | 57 | 18 |
| TMZ | 61.5 | 27 |
| RG5579 + TMZ | 125.5 | 159 |

The results of the in vivo studies are summarized in Table 7 and illustrate the substantial improvement of overall survival following treatment with both anti-miR-10b compound and TMZ. RG5461 and RG5658 improved overall median survival in combination with TMZ treatment. RG5579 treatment increased overall median survival, both as a single agent and in combination with TMZ treatment.

TABLE 7
Anti-miR-10b Efficacy in GBM Models

| Model | Treatment | RG384124 | RG5579 | RG5461 | RG5658 |
|---|---|---|---|---|---|
| LN229 | Intratumoral | No | 14% median | No | No |
| Orthotopic | anti-miR- | survival | survival | survival | survival |
| Model | 10b | benefit | increase | benefit | benefit |
| LN229 | Intratumoral | Not tested | 159% | 59% | 71% |
| Orthotopic | anti-miR- | | median | median | median |
| Model | 10b + TMZ | | survival | survival | survival |
| | | | increase | increase | increase |

In preliminary safety assays, each of the three compounds in Table 7 was found to be well-tolerated following either systemic or intratumoral administration.

### Example 4: De-Repression of miR-10b Downstream Genes

To evaluate the on-target pharmacodynamic effects of treatment with anti-miR-10b, 18 genes that are direct targets of miR-10b were identified by next generation sequencing, and the expression of each of these genes following anti-miR-10b treatment was measured. The 18 genes were: ATXN2, ATXN7, BCL6, BDNF, CRLF3, DAZAP1, DVL3, FXR2, GATAD2A, GCLM, GTF2H1, INO80D, MIEF1, NCOA6, NFE2L1, PDE4A, SMAD2, and TET2.

LN229 cells were treated with a concentration of RG5579 ranging from 2 to 500 nM. After 24 hours, RNA was isolated and the mRNA levels of the 18 genes targeted were measured and averaged to provide a pharmacodynamic signature score (PD Signature Score), represented as Log 2 fold-change (Log 2FC) relative to mock-transfection. Treatment with RG5579 resulted in a dose-dependent de-repression of the PD signature in LN229 cells. Similarly, in the orthotopic LN229 GBM tumor model, treatment with RG5579 at doses of 40 or 80 ug resulted in de-repression of the PD signature.

Similar studies were conducted with RG5461 and RG5658, using a PD signature of 10 genes (non-overlapping with the 18-gene PD signature above). Treatment with either compound resulted in a dose-dependent de-repression of a 10-gene PD signature in LN229 cells.

These data demonstrate that treatment with anti-miR-10b de-represses direct targets of miR-10b.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, GENBANK® accession numbers, and the like) cited in the present application is specifically incorporated herein by reference in its entirety.

1. A compound comprising a modified oligonucleotide, wherein the modified oligonucleotide consists of 21 linked nucleosides and the structure of the modified oligonucleotide is:
(SEQ ID NO: 2)
5′- C_{K}A_{K}A_{K}AU_{K}U_{K}C_{K}GG_{K}U_{E}U_{E}C_{E}U_{E}A_{E}C_{E}A_{E}G_{E}G_{E}G_{E}U_{E}A_{E} -3′
wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein each U is independently selected from a non-methylated uracil and a 5-methyluracil; wherein each C is independently selected from a non-methylated cytosine and a 5-methylcytosine; and wherein each linkage is a phosphorothioate linkage; or a pharmaceutically acceptable salt thereof.

2. The compound of claim 1, wherein the modified oligonucleotide consists of 21 linked nucleosides and the structure of the modified oligonucleotide is:
(SEQ ID NO: 2)
5′- C_{K}A_{K}A_{K}AU_{K}U_{K}C_{K}GG_{K}^{m}U_{E}^{m}U_{E}^{m}C_{E}^{m}U_{E}A_{E}^{m}C_{E}A_{E}G_{E}G_{E}G_{E}^{m}U_{E}
A_{E}-3′
wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein a “^{m}U” is a 5-methyluracil and “U” is a non-methylated uracil; wherein a “^{m}C” is a 5-methylcytosine and “C” is a non-methylated cytosine; and wherein each linkage is a phosphorothioate linkage; or a pharmaceutically acceptable salt thereof.

3. A compound comprising a modified oligonucleotide, wherein the modified oligonucleotide consists of 21 linked nucleosides and the structure of the modified oligonucleotide is:
(SEQ ID NO: 2)
5′-C_{K}A_{K}A_{E}A_{E}U_{K}U_{E}C_{E}G_{K}G_{E}U_{E}U_{K}C_{E}U_{E}A_{K}C_{E}A_{E}G_{E}G_{E}G_{E}U_{E}A_{E}-3′
wherein nucleosides followed by subscript “E” are 2′-O-methoxyethyl nucleosides, nucleosides followed by subscript “K” are S-cEt nucleosides, and nucleosides without a subscript are β-D-deoxyribonucleotides; wherein each U is independently selected from a non-methylated uracil and a 5-methyluracil; wherein each C is independently selected from a non-methylated cytosine and a 5-methylcytosine; and wherein each linkage is a phosphorothioate linkage; or a pharmaceutically acceptable salt thereof.

4. The compound of claim 3, wherein the modified oligonucleotide consists of 21 linked nucleosides and the structure of the modified oligonucleotide is:
(SEQ ID NO: 2)
5′-C_{K}A_{K}A_{E}A_{E}U_{K}^{m}U_{E}^{m}C_{E}G_{K}G_{E}^{m}U_{E}U_{K}^{m}C_{E}^{m}U_{E}A_{K}^{m}C_{E}A_{E}G_{E}G_{E}G_{E}^{m}U_{E}
A_{E}-3′
wherein nucleosides followed by subscript “E” are 2′-O-methoxyethyl nucleosides, and nucleosides followed by subscript “K” are S-cEt nucleosides; wherein a “^{m}U” is a 5-methyluracil and “U” is a non-methylated uracil; wherein a “^{m}C” is a 5-methylcytosine; and wherein each linkage is a phosphorothioate linkage; or a pharmaceutically acceptable salt thereof.

5. A compound comprising a modified oligonucleotide consisting of 9 linked nucleosides, wherein the modified oligonucleotide comprises the structure:
5′-U_{K}A_{K}C_{M}A_{F}G_{F}G_{F}G_{M}U_{K}A_{K}-3′
wherein nucleosides followed by subscript “K” are S-cEt nucleosides, nucleosides followed by subscript “M” are 2′-O-methyl nucleosides, and nucleosides followed by subscript “F” are 2′-fluoro nucleosides; wherein each U is independently selected from a non-methylated uracil and a 5-methyluracil; wherein each C is independently selected from a non-methylated cytosine and a 5-methylcytosine; and wherein each internucleoside linkage is a phosphorothioate linkage; or a pharmaceutically acceptable salt thereof.

6. The compound of claim 5, wherein the modified oligonucleotide consists of 9 linked nucleosides and the structure of the modified oligonucleotide is:
5′-U_{K}A_{K}C_{M}A_{F}G_{F}G_{F}G_{M}U_{K}A_{K}-3′
wherein nucleosides followed by subscript “K” are S-cEt nucleosides, nucleosides followed by subscript “M” are 2′-O-methyl nucleosides, and nucleosides followed by subscript “F” are 2′-fluoro nucleosides; wherein a “U” is a non-methylated uracil; wherein a “C” is a non-methylated cytosine; wherein a superscript “O” indicates a phosphodiester linkage and each other internucleoside linkage is a phosphorothioate linkage; or a pharmaceutically acceptable salt thereof.

7. The compound of claim 1, wherein the compound consists of the modified oligonucleotide, or a pharmaceutically acceptable salt thereof.

8. The compound of claim 1, wherein the pharmaceutically acceptable salt is a sodium salt.

9. A pharmaceutical composition comprising a compound of claim 1, and a pharmaceutically acceptable diluent.

10. The pharmaceutical composition of claim 9, wherein the pharmaceutically acceptable diluent is an aqueous solution.

11. The pharmaceutical composition of claim 10, wherein the aqueous solution is a saline solution.

12. A pharmaceutical composition comprising a compound of claim 1, which is a lyophilized composition.

13. A pharmaceutical composition consisting essentially of a compound of claim 1 in a saline solution.

14. A method of treating glioma, comprising administering to a subject having glioma a compound of claim 1.

15. The method of claim 14, wherein the glioma is diffuse astrocytoma, anaplastic astrocytoma, oligodendroglioma, anaplastic oligodendroglioma, diffuse midline glioma, or glioblastoma.

16. The method of claim 14, wherein the compound or pharmaceutical composition is administered intratumorally.

17. The method of claim 15, wherein the diffuse astrocytoma comprises an isocitrate dehydrogenase (IDH) gene mutation; or wherein the anaplastic astrocytoma comprises an isocitrate dehydrogenase (IDH) gene mutation; or wherein the oligodendroglioma comprises an isocitrate dehydrogenase (IDH) gene mutation and a deletion of chromosomal arms 1p and 19q; or wherein the anaplastic oligodendroglioma comprises an isocitrate dehydrogenase (IDH) gene mutation and a deletion of chromosomal arms 1p and 19q; or wherein the diffuse midline glioma comprises a comprises a histone H3 (H3) K27M mutation: or wherein the glioblastoma does not comprise an isocitrate dehydrogenase (IDH) gene mutation: or wherein the glioblastoma comprises an isocitrate dehydrogenase (IDH) gene mutation.

18. (canceled)

19. (canceled)

20. (canceled)

21. (canceled)

22. (canceled)

23. (canceled)

24. The method of claim 14, wherein the glioma is a recurrent glioma.

25. The method of claim 17, wherein the isocitrate dehydrogenase (IDH) gene mutation is an IDH1 or IDH2 gene mutation.

26. The method of claim 14, wherein following administration of the compound or pharmaceutical composition, tumor size is reduced and/or tumor number is reduced.

27. The method of claim 14, wherein the administering of the compound increases progression-free survival of the subject; and/or wherein the administering of the compound increases overall survival time of the subject; and/or wherein the administering of the improves the subject's quality of life.

28. (canceled)

29. (canceled)

30. The method of claim 14, comprising administering at least one additional anti-cancer therapy.

31. The method of claim 30, wherein the at least one additional therapy is selected from surgical resection, radiotherapy, tumor treating fields, and one or more chemotherapeutic agents.

32. The method of claim 31, wherein the chemotherapeutic agent is selected from carmustine, temozolomide, and bevacizumab.

33. (canceled)

34. (canceled)


SEQUENCE LISTING (19 sequences)

SEQ ID NO: 1 - RNA, 23 nt, Homo sapiens
uacccuguagaaccgaauuugug

SEQ ID NO: 2 - RNA, 21 nt, Artificial sequence
  Synthetic oligonucleotide
caaauucgguucuacagggua

SEQ ID NO: 3 - DNA, 23 bp, Artificial sequence
  Synthetic oligonucleotide
cacaaattcggttctacagggta

SEQ ID NO: 4 - DNA, 19 bp, Artificial sequence
  Synthetic oligonucleotide
  misc_feature (9)..(9): RNA
  misc_feature (11)..(11): RNA
  misc_feature (18)..(18): RNA
aattcggtucuacagggua

SEQ ID NO: 5 - DNA, 19 bp, Artificial sequence
  Synthetic oligonucleotide
  misc_feature (8)..(8): RNA
  misc_feature (9)..(9): RNA
  misc_feature (11)..(11): RNA
  misc_feature (18)..(18): RNA
aattcgguucuacagggua

SEQ ID NO: 6 - DNA, 19 bp, Artificial sequence
  Synthetic oligonucleotide
  misc_feature (3)..(3): RNA
  misc_feature (11)..(11): RNA
  misc_feature (18)..(18): RNA
aautcggttcuacagggua

SEQ ID NO: 7 - DNA, 19 bp, Artificial sequence
  Synthetic oligonucleotide
  misc_feature (3)..(3): RNA
  misc_feature (9)..(9): RNA
aautcggtuctacagggta

SEQ ID NO: 8 - DNA, 21 bp, Artificial sequence
  Synthetic oligonucleotide
  misc_feature (11)..(11): RNA
  misc_feature (20)..(20): RNA
caaattcggtuctacagggua

SEQ ID NO: 9 - DNA, 21 bp, Artificial sequence
  Synthetic oligonucleotide
  misc_feature (6)..(6): RNA
caaatucggttctacagggta

SEQ ID NO: 10 - DNA, 21 bp, Artificial sequence
  Synthetic oligonucleotide
  misc_feature (5)..(5): RNA
caaautcggttctacagggta

SEQ ID NO: 11 - DNA, 21 bp, Artificial sequence
  Synthetic oligonucleotide
  misc_feature (5)..(5): RNA
  misc_feature (11)..(11): RNA
caaautcggtuctacagggta

SEQ ID NO: 12 - DNA, 21 bp, Artificial sequence
  Synthetic oligonucleotide
  misc_feature (5)..(6): RNA
caaauucggttctacagggta

SEQ ID NO: 13 - DNA, 23 bp, Artificial sequence
  Synthetic oligonucleotide
  misc_feature (8)..(8): RNA
cacaaatucggttctacagggta

SEQ ID NO: 14 - DNA, 23 bp, Artificial sequence
  Synthetic oligonucleotide
  misc_feature (7)..(7): RNA
cacaaautcggttctacagggta

SEQ ID NO: 15 - DNA, 23 bp, Artificial sequence
  Synthetic oligonucleotide
  misc_feature (7)..(7): RNA
  misc_feature (13)..(13): RNA
cacaaautcggtuctacagggta

SEQ ID NO: 16 - DNA, 23 bp, Artificial sequence
  Synthetic oligonucleotide
  misc_feature (7)..(8): RNA
cacaaauucggttctacagggta

SEQ ID NO: 17 - RNA, 22 nt, Artificial sequence
  Synthetic oligonucleotide
cuacaggguaaacuacagggua

SEQ ID NO: 18 - RNA, 20 nt, Artificial sequence
  Synthetic oligonucleotide
uacaggguaaauacagggua

SEQ ID NO: 19 - DNA, 17 bp, Artificial sequence
  Synthetic oligonucleotide
  misc_feature (1)..(2): RNA
  misc_feature (7)..(7): RNA
uucggtuctacagggta